(19) United States
(12) Patent Application Publication
(10) Pub. No.: US 2025/0340929 A1
(43) Pub. Date: Nov. 6, 2025

(54) METHODS AND COMPOSITIONS FOR IMAGING RNA AND PROTEIN TARGETS IN BIOLOGICAL SPECIMENS

(71) Applicant: Howard Hughes Medical Institute, Chevy Chase, MD (US)

(72) Inventors: Zhe J. Liu, Ashburn, VA (US); Valentina Gandin, Ashburn, VA (US)

(21) Appl. No.: 19/199,837

(22) Filed: May 6, 2025

Related U.S. Application Data
(60) Provisional application No. 63/643,155, filed on May 6, 2024.

(51) Int. Cl. C12Q 1/6818; C12Q 1/6804; C12Q 1/6806; C12Q 1/6825; C12Q 1/6841

(52) U.S. Cl. CPC C12Q 1/6818; C12Q 1/6804; C12Q 1/6806; C12Q 1/6825; C12Q 1/6841

(57) ABSTRACT

This document describes modified methods and compositions for cycle hybridization chain reaction (“cycleHCR”), which represents a highly versatile and precise method for studying RNA and protein spatial distribution in biological specimens.

### TECHNICAL FIELD

This disclosure generally relates to methods and compositions for imaging RNA and protein targets in biological specimens.

### BACKGROUND

In situ hybridization based on the hybridization chain reaction (HCR) has addressed long-standing challenges that impeded imaging of mRNA expression in diverse organisms, offering a unique combination of multiplexing, quantitation, sensitivity, resolution and versatility. Here, these capabilities are improved upon even further to create methods referred to as cycleHCR.

### SUMMARY

The inherent limitations of fluorescence microscopy, notably the restricted number of color channels, have long constrained comprehensive spatial analysis in biological specimens. Here, we introduce cycleHCR technology that leverages multicycle DNA barcoding and Hybridization Chain Reaction (HCR) to surpass the conventional color barrier. cycleHCR facilitates high-specificity, single-shot imaging per target for RNA and protein species within thick specimens, mitigating the molecular crowding issues encountered with other imaging-based spatial omics techniques. We demonstrate whole-mount transcriptomics imaging of 254 genes within an E6.5˜7.0 mouse embryo, achieving precise three-dimensional gene expression and cell fate mapping across a specimen depth of ˜310 μm. Utilizing expansion microscopy alongside protein cycleHCR, we unveil the complex network of 10 subcellular structures in primary mouse embryonic fibroblasts. Furthermore, in mouse hippocampal slice, we image 8 protein targets and profile the transcriptome of 120 genes, uncovering complex gene expression gradients and cell-type specific nuclear structural variances. cycleHCR provides a unifying framework for multiplex RNA and protein imaging, offering a quantitative solution for elucidating spatial regulations in deep tissue contexts for research and potentially diagnostic applications.

In one aspect, articles of manufacture are provided. Such articles of manufacture typically include a plurality of pairs of primary probes and a plurality of pairs of readout probes. Generally, each pair of primary probes includes left primary probe and a right primary probe, wherein: the left primary probe includes a forward PCR sequence, a left target complementary sequence, a left barcode sequence, and a reverse PCR sequence, and the right primary probe includes the forward PCR sequence, a right barcode sequence, a right target complementary sequence, and the reverse PCR sequence. Generally, each pair of readout probes includes left readout probe and a right readout probe, wherein the left readout probe includes a left HCR initiation sequence and a sequence that is complementary to the left barcode sequence and the right readout probe includes a right HCR initiation sequence and a sequence that is complementary to the right barcode sequence.

In some embodiments, such articles of manufacture further include a first and a second HCR amplifier sequence, wherein each of the first and the second HCR amplifier sequences include a sequence that forms a hairpin and a detectable label, wherein a portion of the first HCR amplifier sequence and a portion of the second HCR amplifier sequence have complementarity to one another.

In some embodiments, such articles of manufacture further include one or more detectable labels (e.g., fluorophores or pairs of fluorophores). In some embodiments, such articles of manufacture further include reagents necessary for hybridization chain reaction (HCR) to occur. In some embodiments, such articles of manufacture further include one or more reagents necessary for fixing the biological specimen. In some embodiments, such articles of manufacture further include one or more reagents necessary for stripping HCR products and probes from the biological specimen.

In another aspect, methods of spatially mapping a target RNA in a biological specimen are provided. Such methods typically include contacting the biological specimen with a pair of primary probes, wherein the pair of primary probes includes a left primary probe and a right primary probe, wherein the left primary probe includes a forward PCR sequence, a left target complementary sequence, a left barcode sequence, and a reverse PCR sequence, and the right primary probe includes the forward PCR sequence, a right barcode sequence, a right target complementary sequence, and the reverse PCR sequence; contacting the biological specimen with a pair of readout probes, wherein the pair of readout probes includes a left readout probe and a right readout probe, wherein: the left readout probe includes a left HCR initiation sequence and a sequence that is complementary to the left barcode sequence and the right readout probe includes a right HCR initiation sequence and a sequence that is complementary to the right barcode sequence; contacting the biological specimen with a first and a second HCR amplifier sequence, wherein each of the first and the second HCR amplifier sequences include a sequence that forms a hairpin and a detectable label, wherein a portion of the first HCR amplifier sequence and a portion of the second HCR amplifier sequence have complementarity to one another; exposing the biological specimen to conditions under which hybridization chain reaction (HCR) occurs to produce labeled HCR products; and imaging the labeled HCR products in the biological specimen, thereby spatially mapping the target RNA in the biological specimen.

In some embodiments, the biological specimen is cultured cells or tissue. In some embodiments, the biological specimen is fixed. In some embodiments, the biological specimen is permeabilized, gelled, contacted with a protease, stained (e.g., DAPI), washed, or combinations thereof.

In some embodiments, the methods further include stripping the HCR products and the readout probes from the biological specimen and repeating the contacting and exposing steps with a different pair of readout probes.

In some embodiments, the biological specimen is contacted with a plurality of pairs of primary probes. In some embodiments, the plurality of pairs of primary probes is included within a primary probe library.

In some embodiments, the detectable label is a fluorophore or one member of a pair of fluorophores. In some embodiments, the methods further include evaluating the quality of the RNA in the biological specimen. In some embodiments, the spatially mapping is three-dimensional.

In some embodiments, the method is high-throughput. In some embodiments, the method is fully automated.

In still another aspect, articles of manufacture are provided. Such articles of manufacture typically include a pair of docking sequences and a pair of gel anchoring probes. Generally, the pair of docking sequences include a first docking sequence and second docking sequence, wherein at least a portion of the first docking sequence and the sequence docking sequence is identical or essentially identical. Generally, the pair of gel anchoring probes includes a first gel anchoring probe and a second gel anchoring probe, wherein a portion of the first gel anchoring probe and second gel anchoring probe are complementary to the portion of the first and second docking sequence that are identical or essentially identical, wherein the first gel anchoring probe and the second gel anchoring probe each include a right barcode sequence and a left barcode sequence.

In some embodiments, such articles of manufacture further include a pair of readout probes, wherein the pair of readout probes includes a left readout probe and a right readout probe, wherein the left readout probe includes a left HCR initiation sequence and a sequence that is complementary to the left barcode sequence and the right readout probe includes a right HCR initiation sequence and a sequence that is complementary to the right barcode sequence.

In some embodiments, such articles of manufacture further include a first and a second HCR amplifier sequence, wherein each of the first and the second HCR amplifier sequences include a sequence that forms a hairpin and a detectable label, wherein a portion of the first HCR amplifier sequence and a portion of the second HCR amplifier sequence have complementarity to one another.

In some embodiments, such articles of manufacture further include one or more proteins that bind one or more targets in the biological specimen. In some embodiments, such articles of manufacture further include one or more detectable labels (e.g., fluorophores or pairs of fluorophores).

In some embodiments, such articles of manufacture further include reagents necessary for hybridization chain reaction (HCR) to occur. In some embodiments, such articles of manufacture further include one or more reagents necessary for fixing the biological specimen. In some embodiments, such articles of manufacture further include one or more reagents necessary for stripping HCR products and probes from the biological specimen.

In some embodiments, such articles of manufacture further include a linker such as, without limitation, a light-activated oYo linker.

In some embodiments, the pair of gel anchoring probes further includes at least one 5′ acrydite modifications.

In still another aspect, methods of spatially mapping a target protein in a biological specimen are provided. Such methods typically include contacting the biological specimen with an antibody complex that binds specifically to the target protein, wherein the antibody complex includes two identical or essentially identical docking sequences covalently attached thereto, wherein the antibody complex further includes two identical or essentially identical gel anchoring probes hybridized to the two docking sequences, wherein each of the gel anchoring probes further includes a left barcode sequence and a right barcode sequence; immobilizing the biological specimen including the antibody complex bound to the target protein in a gel via either or both of the gel anchoring probes; contacting the biological specimen with a pair of readout probes, wherein the pair of readout probes includes a left readout probe and a right readout probe, wherein the left readout probe includes a left HCR initiation sequence and a sequence that is complementary to the left barcode sequence and the right readout probe includes a right HCR initiation sequence and a sequence that is complementary to the right barcode sequence; contacting the biological specimen with a first and a second HCR amplifier sequence, wherein each of the first and the second HCR amplifier sequences include a sequence that forms a hairpin and a detectable label, wherein a portion of the first HCR amplifier sequence and a portion of the second HCR amplifier sequence have complementarity to one another; exposing the biological specimen to conditions under which hybridization chain reaction (HCR) occurs to produce HCR products; and imaging the HCR products in the biological specimen, thereby spatially mapping the target protein in the biological specimen.

In some embodiments, the biological specimen is cultured cells or tissue. In some embodiments, the biological specimen is fixed. In some embodiments, the biological specimen is permeabilized, gelled, contacted with a protease, staining, washing, and combinations thereof.

In some embodiments, the docking sequence is attached to the antibody via a linker (e.g., a light-activated oYo linker). In some embodiments, the gel anchoring probes include at least one 5′ acrydite modifications.

In some embodiments, the methods further include stripping the HCR products and the readout probes from the biological specimen and repeating the contacting and exposing steps with a different pair of readout probes.

In some embodiments, the method is high-throughput. In some embodiments, the method is fully automated.

In one aspect, methods of spatially mapping a target RNA in a biological specimen are provided. Such methods typically include contacting the biological specimen with a pair of primary probes, wherein the pair of primary probes includes a left primary probe and a right primary probe, wherein: the left primary probe includes a forward PCR sequence, a left target complementary sequence, a left barcode sequence, and a reverse PCR sequence, and the right primary probe includes the forward PCR sequence, a right barcode sequence, a right target complementary sequence, and the reverse PCR sequence; contacting the biological specimen with a pair of readout probes, wherein the pair of readout probes includes a left readout probe and a right readout probe, wherein the left readout probe includes a left HCR initiation sequence and a sequence that is complementary to the left barcode sequence and the right readout probe includes a right HCR initiation sequence and a sequence that is complementary to the right barcode sequence; contacting the biological specimen with a first and a second HCR amplifier sequence each including a fluorophore (or each including one member of a pair of fluorophores); exposing the biological specimen to conditions under which hybridization chain reaction (HCR) occurs to produce HCR products; and imaging the HCR products in the biological specimen, thereby spatially mapping the target RNA in the biological specimen.

In some embodiments, the biological specimen is cultured cells or tissue. In some embodiments, the biological specimen is fixed. In some embodiments, the biological specimen is permeabilized, gelled, contacted with a protease, stained (e.g., DAPI), washed, or combinations thereof. In some embodiments, the mapping is three-dimensional.

In some embodiments, the methods further include evaluating the quality of the RNA in the biological specimen. In some embodiments, the methods further include stripping the HCR products and the readout probes from the biological specimen and repeating the contacting and exposing steps with a different pair of readout probes.

In some embodiments, the biological specimen is contacted with a plurality of pairs of primary probes. In some embodiments, the plurality of pairs of primary probes is included within a primary probe library.

In some embodiments, the method is high-throughput. In some embodiments, the method is fully automated.

In another aspect, methods of spatially mapping a target protein in a biological specimen are provided. Such methods typically include: contacting the biological specimen with an antibody complex that binds specifically to the target protein, wherein the antibody complex includes two identical docking sequences covalently attached thereto, wherein the antibody complex further includes two identical gel anchoring probes hybridized to the two docking sequences, wherein each of the gel anchoring probes further includes a left barcode sequence and a right barcode sequence; immobilizing the biological specimen including the antibody complex bound to the target protein in a gel via either or both of the gel anchoring probes; contacting the biological specimen with a pair of readout probes, wherein the pair of readout probes includes a left readout probe and a right readout probe, wherein the left readout probe includes a left HCR initiation sequence and a sequence that is complementary to the left barcode sequence and the right readout probe includes a right HCR initiation sequence and a sequence that is complementary to the right barcode sequence; contacting the biological specimen with a first and a second HCR amplifier sequence each including a fluorophore; exposing the biological specimen to conditions under which hybridization chain reaction (HCR) occurs to produce HCR products; and imaging the HCR products in the biological specimen, thereby spatially mapping the target protein in the biological specimen.

In some embodiments, the biological specimen is tissue. In some embodiments, the biological specimen is fixed. In some embodiments, the biological specimen is permeabilized, gelled, contacted with a protease, staining, washing, and combinations thereof.

In some embodiments, the docking sequence is attached to the antibody via a linker. In some embodiments, the linker is a light-activated oYo linker.

In some embodiments, the gel docking sequence includes one or more 5′ acrydite modifications.

In some embodiments, further including stripping the HCR products and the readout probes from the biological specimen and repeating the contacting and exposing steps with a different pair of readout probes.

In some embodiments, the method is high-throughput. In some embodiments, the method is fully automated.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

The patent or application file contains at least one drawing executed in color. Copies of this patent or patent application publication with color drawings will be provided by the Office upon request any payment of the necessary fee.

FIGS. 1A-1G. cycleHCR RNA imaging in thick tissues.

(1A) To target each RNA sequence with high specificity, we engineered DNA probe libraries with 10 to 25 pairs of 45-bp probes. Each pair consists of left and right probes with unique 14-bp barcode sequences for binding left (L) and right (R) readout probes with split HCR initiators, triggering the hybridization chain reaction (HCR). The arrowhead indicates the 3′ end of RNA or DNA.

(1B) The HCR amplification occurs only with perfectly matched L and R readout probe pairs. Mismatched probes, single probes, or absence of probes prevent HCR initiation, minimizing false positives. Target: Trim6; L2+R2 (561). Scale bars: 10 μm.

(1C) Quantification of images shown in (B) is performed by comparing the HCR signal to the nuclear DAPI signal for normalizing HCR signals to the number of cells per region of interest (n=30). The error bars represent standard deviations. A one-way ANOVA was conducted to assess significant differences across all groups, followed by pairwise p-values calculated using Bonferroni tests, with the no-barcode group serving as the reference. n.s., non-significant (p>0.05); ****, p<0.0001.

(1D) The mixing of 30 left and 30 right probes, each with unique divergent sequences, allows for the creation of up to 900 distinct barcodes for each fluorescence channel. Images are acquired using three separate fluorescence channels (488 nm, 561 nm, 640 nm), each channel harboring orthogonal B4, B2, and B3 HCR initiators.

(1E) This panel demonstrates cycleHCR labeling specificity in thick specimens for three distinct cell-type marker genes. Through sequential three-round cycleHCR imaging, we show specific labeling changes for Slc1a3 (L1+R1 at 640 nm, marking Bergmann glia cells), Rgs8 (L1+R2 at 640 nm, marking Purkinje cells), and Cbln3 (L2+R1 at 640 nm, marking the granule layer) in cerebellum specimens approximately 200 μm thick. 3D images were rendered using the normal shading mode in Imaris. Scale bar: 50 μm.

(1F) Two cycleHCR codebooks were used to measure the expression of the same set of 50 genes in mouse embryonic stem cells. Spots per cell (black dots) are plotted with linear regression in the red line.

(1G) Evaluation of spot detection over axial depth using a 25× silicone oil immersion objective. The left panel shows the relationship between spot intensity and depth, indicating relatively stable spot intensity up to 400 μm, with less than 10% reduction beyond 400 μm. The shaded region reflects standard deviation. The middle panel presents raw data acquired at a 513 μm depth in both sparsely and densely labeled regions, while the right panel displays spot detection marked with red circles overlaid on the raw image. Smaller red circles indicate the centers of these molecules are in other z slices. Scale bar: 20 μm.

FIGS. 2A-2E. Whole-embryo transcriptomics imaging across a depth of ˜310 μm.

(2A) Orthogonal xy and yz views of a 10-color composite image displaying the expression patterns of 10 genes (Lhx1, Dhmt3al, Pim2, Cldn6, Tex9.1, Amot, Eomes, Cubn, Col4a1, and Cdh5) out of 254 genes imaged by cycleHCR in an E6.5-7.0 mouse embryo. The slice views were rendered by the maximal intensity projection (MIP) view using Imaris software. Scale bar: 50 μm.

(2B) Random colored masks for 11,029 cells segmented by Cellpose and rendered by ORS Dragonfly software.

(2C) An inverted raw image showing the detection of Pim2 mRNA transcripts at the single-molecule level (black spots), with single-molecule localizations encircled in red.

(2D) A zoomed-in view of the region marked in (C), providing enhanced detail on the accuracy of single-molecule localization. Smaller red circles indicate the centers of these molecules are in other z slices. Scale bar: 10 μm.

(2E) 3D spatial gene expression maps for Pim2 mRNA, with spots assigned to individual cells based on Cellpose masks that are slightly dilated compared to DAPI labeled nucleus. Cells in the resulting xy and yz views are color-coded based on transcript counts indicated by the provided color map. Scale bar: 50 μm.

FIGS. 3A-3B. 3D cell-fate map reconstruction.

(3A) UMAP analysis and gene clustering on single-cell transcript counts for 186 expressed genes detected by cycleHCR identify 9 distinct cell clusters. The number of cells within each cluster is indicated in the left panel. The right panel shows the clustering of the 186 genes, with 5 representative genes for individual clusters highlighted below. The color map represents the min-max scaled expression for each gene across clusters.

(3B) A 3D cell-map, color-coded per (3A), displays the center of mass for each cell with a surrounding mesh for nuclear outlines. The left panel shows all 9 clusters. Upper middle panels provide views of clusters C1-C5 from the same and a ˜90-degree rotated orientation, and the right panel displays the extra-embryonic layers (C5, C7, C8, and C9) alongside two closely situated intra-embryo clusters (C2 and C3). Lower panels detail the spatial distribution of outer layer clusters (C6-C9). Expression patterns for 18 selected genes are shown in the right and bottom zoom-out images by overlapping raw RNA localization patterns (dark green) onto the embryo mask. Images were rendered using the Napari Python package. Scale bars: 50 μm.

FIGS. 4A-4F. 3D geometrical mapping of gradients and heterogeneity in gene expression.

(4A) Cell cartesian coordinates (xyz) were transformed along three biologically relevant axes in relation to the embryo structure: the proximal-distal axis (defined by distance along the midline), the anterior-posterior axis (defined by the angle between each cell, the midline, and the posterior line), and the radial axis (defined by the distance of each cell from the midline). Cells were divided into left and right sides based on the positive and negative values along the anterior-posterior axis. Cells are color-coded per FIG. 3A.

(4B) Cells on the left and right symmetric sides of the embryo occupy distinct z-positions during imaging.

(4C) Overall gene expression is highly correlated between the left and right sides of the embryo, suggesting that imaging depth does not substantially affect cycleHCR gene expression measurements.

(4D) UMAP imputation revealed that Brachyury (T) is over-represented in clusters C2, C3, and C4.

(4E) Top: Genes are ranked by their slopes across three independent biological axes for each cluster, from positive-ascending to negative-descending, with three selected panels displayed for clusters C2 (distal-proximal axis), C3 (distal-proximal axis), and C4 (anterior-posterior axis). Bottom: Color-coded cell positions and 3D gene expression maps for selected genes are displayed in the lower panel. Single-cell gene expression heatmaps for selected genes show gradients and substantial heterogeneity even among cells with similar gradient trends. The slope data for all 9 clusters along the 3 axes are in Table S5.

(4F) Gene co-expression analysis revealed distinct co-expression patterns for each cluster, with a zoomed-in view of the co-expression heatmap for cluster C2. This heatmap distinguishes gene pairs with the same expression gradient along the given axis but with varying correlations in single-cell expression, as illustrated by the three insets on the right with colored squares indicating co-expression levels.

Scale bars: 50 μm.

FIGS. 5A-5H. Multiplex protein and RNA imaging via cycleHCR in hippocampal slices.

(5A-5D) Hierarchical images displaying structures at various length scales, with the axonal initial segment marker AnkG staining featured in (A). A closer examination of AnkG, astrocyte marker GFAP, and Tiam1 cycleHCR RNA within the boxed region is shown in (B). Further zooming reveals Fibrillarin, SF3A66, H3K4me1, H3K27me3, and GFAP in the boxed region within (B), as depicted in (C). Zoomed-in views of Fibrillarin, SF3A66, H3K27ac, Nup98, and DAPI within the boxed region in (C) are presented in (D). Scale bars: (A-B) 50 μm; (C) 10 μm; (D) 5 μm.

(5E) UMAP analysis of single-cell transcript counts identifies 6 distinct cell clusters in the hippocampus. The number of cells within each cluster is indicated in the panel, with spatial distributions of approximately 16,691 assigned cells shown in the right panel, matching cluster colors in the left panel.

(5F) Top: cells in clusters 1-3 are divided into 9 distinct segments along their well layout middle line from left to right side. Middle: The average transcript counts per cell for 120 genes along Segments 1 to 9 are plotted using random colors, with multiple genes exhibiting peak gene expression at Segment 1, 7 or 8. Bottom: The distribution of linear slopes for all genes was calculated using the first 8 segments. 33 genes display decreased gene expression along segments with k<=−0.15, while 7 genes show increased gene expression along segments with k>=0.15.

(5G) The solute carrier family protein Slc17a6 showed descending gene expression levels along the C1-C3 axis, while the opposite is observed for Slc17a7.

(5H) Boxplots of nuclear antigen amounts (H3K27ac-active chromatin; H3K4me1-active enhancers; nuclear pore complex subunit-Nup98) normalized to inactive chromatin (H3K27me3) intensity per nuclear mask to eliminate potential local staining imbalances. The lower and upper whiskers represent 10% and 90% values; the box represents the range from 25% to 75% percentile; the center line represents the median; the dotted line indicates the mean. The number of cells for each cluster is annotated in (E). A one-way ANOVA was conducted to assess significant differences across all groups, followed by pairwise p-values calculated using Bonferroni tests, using C1 as the reference. n.s., non-significant (p>0.05); **, p<0.01; ****, p<0.0001.

Scale bars (E-G): 50 μm.

FIG. 6. Primary Probe Selection for Single-Shot cycleHCR RNA Imaging.

To achieve high-specificity RNA imaging in a single-shot with cycleHCR, a multistep probe selection strategy is employed for each target transcript. This involves: 1) Sliding window search: utilizing a sliding window, we identify a 92bp sequence optimal for probe design, divided into 45bp segments for the left and right probe pairs, separated by a 2bp gap. 2) Melting temperature (Tm) estimation: The Tm for DNA-RNA probe binding is calculated to ensure that each probe half binds with a Tm above 90° C. High Tm values contribute to the stability of probe-target interactions even under stringent stripping conditions. 3) Final screening for specificity: a final specificity check involves screening for 26 bp junction sequences across the genome sequence. Any probe pair with more than one genomic match is excluded to avoid non-specific targeting. To further illustrate the specificity of split primary probes, we assign the non-specific binding probabilities for both left (P_{LeftBound}) and right (P_{RightBound}) probes. The overall likelihood of coincidental binding to the same non-specific target is calculated as the product of P_{LeftBound} and P_{RightBound}. Given that HCR activation relies on the proximal binding of both probe halves, the probability of non-target HCR initiation is a product of P_{LeftBound}, P_{RightBound}, and the proximity probability (P_{Proximity}), highlighting the strategic approach to ensure specificity.

FIGS. 7A-7D. Automation of cycleHCR procedures.

(7A) The cycleHCR protocol uses a consistent incubation temperature of 32° C. across all steps after primary probe hybridization. Left and right barcoding probes, each carrying split HCR initiators, are hybridized to the primary probe for 30 minutes. Following a washing step, the HCR amplification process is initiated with h1 and h2 hairpins for 1 hour and 30minutes. Post-amplification, a final washing step precedes imaging. A tailored stripping step then selectively removes HCR hairpins and left-right barcodes without disrupting the robust primary probe: RNA interactions, due to their high melting temperatures (>90° C.). The arrowhead indicates the 3′ end of RNA or DNA.

(7B) The fluidic system integrates an air pressure modulator and a flow sensor with Proportional-Integral-Derivative (PID) feedback to ensure precise control over the flow rate and volume of probes, washes, imaging, and h1/h2 solutions through a distribution valve. A 2-to-1 valve allows for real-time mixing of h1 and h2 solutions. These solutions then pass through a temperature-controlled imaging chamber before disposal into a waste collection bottle.

(7C) The preparation process of Left and Right readout mixes utilizes a programmable pipetting robotic arm.

(7D) The cycleHCR imaging setup includes a CSU-W1 spinning disk microscope, equipped with a temperature control system for both the chamber and objective, integrated with the fluidic system. Communication between the microscope and the fluidic system is facilitated via Transistor-to-Transistor Logic (TTL) communication protocol, ensuring synchronized operation between imaging and fluidic operations.

FIGS. 8A-8B. Evaluation of stripping efficiency and stability of primary probes on RNA targets.

(8A) The multicycle stripping and reprobing efficiency of cycleHCR is evaluated by repetitively stripping and reprobing the same RNA target (Trim6; L2+R2 at 561 nm) across eight cycles. For each cycle, imaging data were captured post-stripping and post-HCR to assess the efficiency of probe removal and the consistency of signal amplification. Pairwise p-values were calculated with ANOVA Bonferroni tests by using the cycle1 after HCR group as the reference. n.s., non-significant (p>0.05); ****, p<0.0001.

(8B) The stability of primary probe-RNA interactions was quantitatively evaluated using nuclear segmentation with Cellpose (left panel), enhanced by a custom-trained model, alongside 3D single molecule localization with RS-FISH. RNA spot counts were determined for each cell within 10 fields of view, totaling approximately 2,908 cells. The analysis revealed no significant decrease in signal intensity across the eight cycles (right panel).

Scale bars: 10 μm.

FIGS. 9A-9B. Mitigating photo-crosslinking in cycleHCR imaging using oxygen scavenger.

(9A) To evaluate photo-crosslinking effect, samples following protein cycleHCR targeting histone H3K27me3 underwent imaging at 100% laser power. Then, continuous illumination for 15 seconds was then applied both with and without the presence of oxygen scavenger (O/S) to determine its effect on photo-crosslinking of HCR fluorescent probes to the specimen. Fluorescence images captured after probe stripping allowed for the assessment of photo-crosslinking levels. The presence of O/S substantially reduces photo-crosslinking. The display range for these images is provided above. Scale bars: 20 μm.

(9B) The impact of O/S on reducing photo-crosslinking was quantitatively evaluated by measuring residual fluorescence signals exceeding background levels per cell in 2D slices. The analysis confirms the effectiveness of oxygen scavenger in minimizing undesired photo-crosslinking effects. The error bars represent standard deviations. A one-way ANOVA was conducted to assess significant differences across all groups, followed by pairwise p-values calculated using Bonferroni tests, with the no imaging and O/S condition serving as the reference. n.s., non-significant (p>0.05); ****, p<0.0001.

FIGS. 10A-10D. L+R barcode cross-reactivity evaluation.

(10A) Melting temperatures (Tm) for on-target and cross-hybridization of 230 14-bp L and R barcoding probes. The maximum cross-hybridization Tm is calculated among probes sharing the same split initiator sequence. The arrowhead indicates the 3′ end of DNA.

(10B) Evaluation of cross-reactivity among barcodes using abundant nuclear protein targets (H3K4me1, SF3A66, and fibrillarin) in NIH/3T3 cells. The L barcode was consistent for H3K4me1 and fibrillarin, and the R barcode was consistent for SF3A66. The test involved cycling through all off-target barcodes on the opposite side to assess cross-reactivity in the first 40 cycles, with the final cycle demonstrating on-target detection. A zoom-in composite cycleHCR image is shown on the right. Scale bar: 5 μm.

(10C) Representative images from indicated cycles for all 4 channels. Scale bar: 50 μm.

(10D) Automated imaging analysis pipeline for 3D cell segmentation and calculation of mean intensity above background and nuclear volume for each mask.

FIGS. 11A-11B. cycleHCR barcode cross-reactivity evaluation.

(11A) Mean intensity over background within nuclear masks for 256 cells across 6 fields of view (FOVs) over 41 cycles. Error bars represent 95% confidence intervals. Negligible barcode cross-talks were detected for off-target barcodes across all 3 channels. Nonspecific fluorescence background increased approximately 1.7% and 2.8% for the 488 nm and 561 nm channels, respectively, while the background for the 640 nm channel remained stable throughout all cycles. This suggests that background increases are likely attributable to autofluorescence. The error bars represent 95% confidence interval.

(11B) Total nuclear volume, measured in number of voxels, for 256 cells across 6 field of views (FOVs) across all 41 cycles. The lower and upper whiskers represent standard deviation (SD); the box shows the range from the 25th to the 75th percentile; the center line represents the median. A one-way ANOVA was conducted to assess significance of differences across all groups.

FIGS. 12A-12C. Amplification fold estimation across 3 color channels.

(12A) To estimate the amplification fold across 3 color channels, we compared conditions without amplification (H1 only) and with typical amplification conditions (H1+H2, 32° C., 90 min) for gel-anchored cycleHCR barcodes targeting H3K4mel (488 nm), SF3A66 (561 nm), and Fibrillarin (640 nm). The left column shows autoscaled images of the H1 only condition. Middle and right columns display images of both H1 only and H1+H2 conditions with identical range settings. Arrowheads indicate the 3′ end of DNA.

(12B) Automated imaging analysis pipeline for 3D cell segmentation and calculation of mean intensity above background for both H1 only and H1+H2 conditions.

(12C) The bar graph represents intensity over background, normalized to the amplification condition (H1+H2). The number of cells and the field-of-views (FOVs) analyzed under each condition is indicated below each bar. Error bars represent the 95% confidence interval. Significance was calculated using a t-test: ****, p<0.0001. Calculated amplification fold for each channel is annotated on the top the graph.

Scale bars: 50 μm.

FIG. 13. Nextflow Orchestrated Image Processing Workflow for cycleHCR.

This figure presents a workflow designed for efficient processing of imaging data. The workflow is orchestrated with Nextflow and includes the following steps: 1) Initial categorization: raw image files, in Nd2 and Tiff formats, are first categorized based on the number of tiles present at each time point. 2) Image stitching: for instances where the number of tiles exceeds one, images undergo stitching via BigStitcher. Conversely, single-tile images bypass this stitching step. 3) RNA localization: RS-FISH is utilized to identify mRNA single molecule localizations 4) Cross-round registration: The BigStream tool calculates cross-round registration matrices, enabling the alignment of images and localization spots across different rounds using DAPI channel data for consistent registration. 5) Cell segmentation: in parallel with registration, cell segmentation is conducted using Cellpose using the reference DAPI channel, employing a custom model refined through a human-in-the-loop method to ensure accurate segmentation of nucleus boundaries. 6) Spot-to-cell assignment: following registration, localizations are assigned to each segmented cell, culminating in a comprehensive localization count per cell for all identified cell masks within the image.

FIGS. 14A-14B. Gene Expression Thresholding and Cell Filtering.

(14A) A histogram showing the distribution of total spot counts for each gene across all cells on a logarithmic scale to accommodate the broad range of expression levels. Genes with fewer than 410 total spots are removed based on Otsu's method, retaining 186 genes for subsequent analysis.

(14B) A histogram presenting the total number of spots assigned per cell, also on a logarithmic scale. Cells with spot counts below 40, approximately 484 cells (5% of the total), were excluded from further analysis.

FIGS. 15A-15C. Gene and Cell Filtering Prior to UMAP Analysis.

(15A) Histogram displaying the distribution of total spot counts for each gene across all cells on a logarithmic scale. Genes with fewer than 12,386 spots were removed based on Li's method, retaining 61 genes for subsequent analysis.

(15B) Histogram illustrating the total number of spots for each cell across the retained 61 genes on a logarithmic scale. Cells with spot counts less than 10, approximately 1,484 (7.5% of total), were excluded from further analysis.

(15C) Heatmap presenting the min-max scaled mean expression per cluster for each gene used in the UMAP analysis.

FIG. 16. Schematic of Protein cycleHCR.

The cycleHCR antibody complex is assembled by 1) conjugating two identical docking sequences to the heavy chain of the antibody using the oYo linker. The sequence is unique to each antibody. 2) Then, two gel anchoring probes with identical sequences are hybridized to the two docking sequences. Each gel anchoring probe contains a 5′ acrydite group for gel integration and a cycleHCR L+R barcode for later cycleHCR imaging readout.

FIG. 17. A Detailed Schematic of Protein cycleHCR.

Upon gel embedding, gel anchoring probes are covalently incorporated into the gel matrix encoding the spatial information of different targets. At this point, the presence of the antibody is not required anymore.

### DETAILED DESCRIPTION

Understanding the spatial organization of molecular components within complex tissue samples is crucial for deciphering the biological regulations that underpin animal development and disease states. Significant advancements in microscopy, tissue clearing and expansion techniques over the past decades have enabled deeper imaging with enhanced clarity and resolution. However, a fundamental limitation of fluorescent imaging remains-it cannot simultaneously image multiple molecular components or color channels due to the restricted availability of labeling dyes and their spectrum overlaps.

Recent advances in high-throughput spatial omics, notably single-molecule in situ hybridization techniques like MERFISH and seqFISH, as well as in situ sequencing methods such as FISSEQ and STARmap, have markedly improved our capacity to spatially decode molecular identities, particularly for RNA species. Despite these innovations, these approaches encounter significant challenges. One such challenge is molecular crowding, which can restrict the decoding capacity for targets that are either abundant or exhibit heterogeneous distributions. Additionally, the necessity to detect dim single molecules with high numerical aperture objectives severely limits axial imaging depth in in situ hybridization-based techniques. Rolling circle amplification (RCA), when combined with expansion microscopy, offers potential solutions to circumvent molecular crowding and enhance imaging depth. However, the inherently low detection efficiency of RCA limits sensitivity and coverage for target detection. Moreover, these methods require precise cross-round registration with nanometer resolution for accurate assignment of detected spots to specific genes, a task that becomes progressively more difficult with increasing specimen size. The lack of empirical ground truth images for verification further complicates the interpretation of RNA target distributions generated by these methods, which rely on cross-round barcoding at the single-molecule level.

The development of split Hybridization Chain Reaction (HCR) techniques has begun to address several critical challenges in deep tissue imaging: First, the proximity binding requirement of split probes to initiate HCR allows for high specificity in the probe hybridization process. This feature enables single-shot imaging of RNA and protein molecules without the need for cross-round decoding and proofing. Second, HCR based signal amplification permits the use of objectives with low numerical apertures and long working distances, enabling reliable deep-tissue detection. Third, the inherent single-shot per target nature of HCR imaging obviates the need for cross-round decoding, making this technique impervious to molecular crowding and suitable for both sparsely and densely labeled targets. Achieving multi-round HCR imaging has been demonstrated through the time-consuming process of primary probe removal by DNase treatment and rehybridization. Currently, this method allows for only one round of imaging every 3-5 days, creating a bottleneck in the workflow and limiting the number of targets that can be effectively examined.

The methods and compositions described herein are intended to address the shortcomings described above and are related to modifications of the HCR techniques. The methods and compositions described herein can be used with any number of biological specimens including, without limitation, cells (e.g., cultured cells) or tissue. It would be understood that, in some instances, a biological specimen can be fixed or gelled (e.g., encased in a gel). In addition, a biological specimen can be permeabilized, contacted with a protease enzyme, stained (e.g., DAPI), and/or washed at various stages of the process as appropriate. The methods and compositions described herein allow for the three-dimensional spatial mapping of cells or tissues.

### Spatial Mapping of One or More Target RNAs

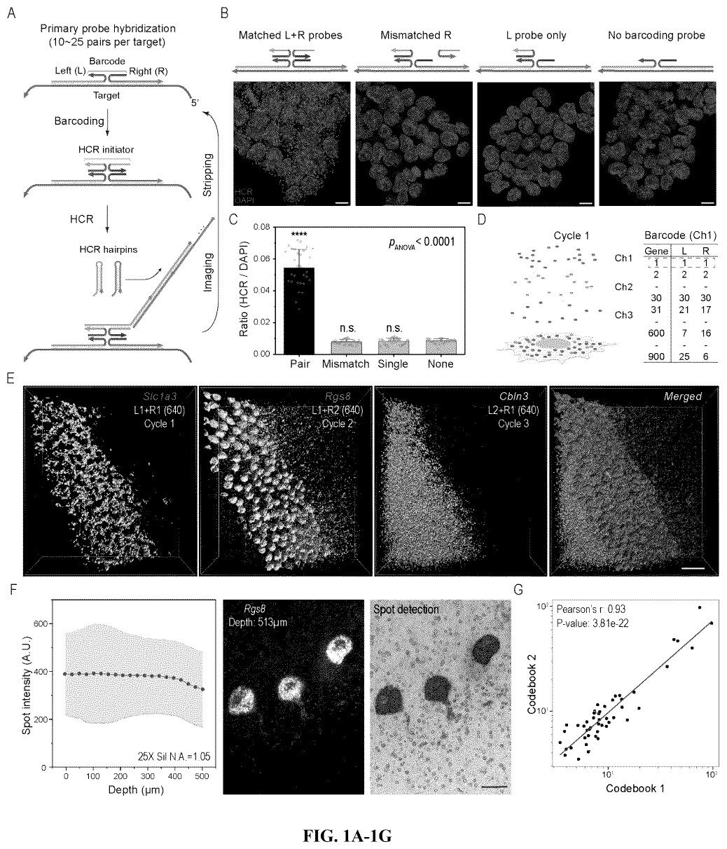

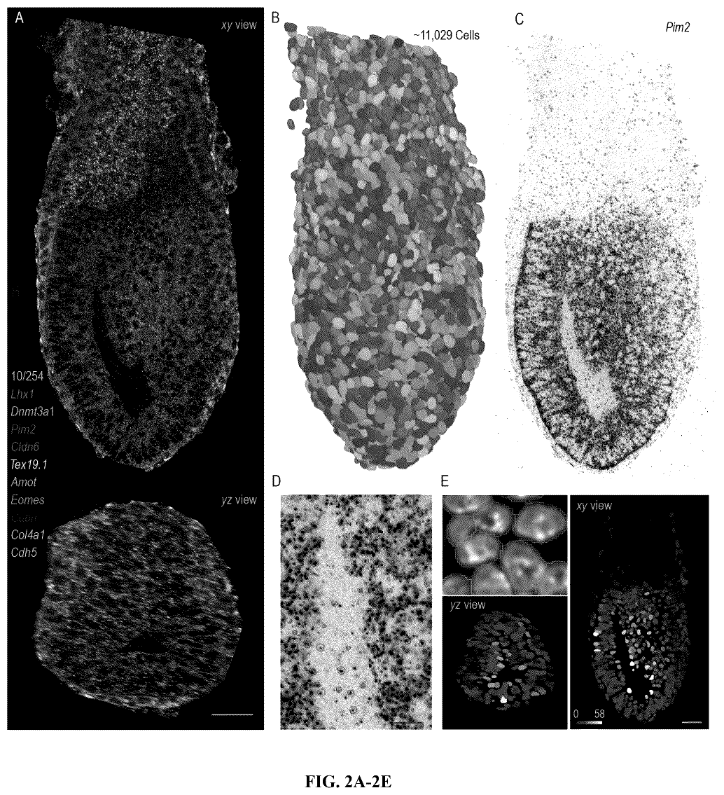

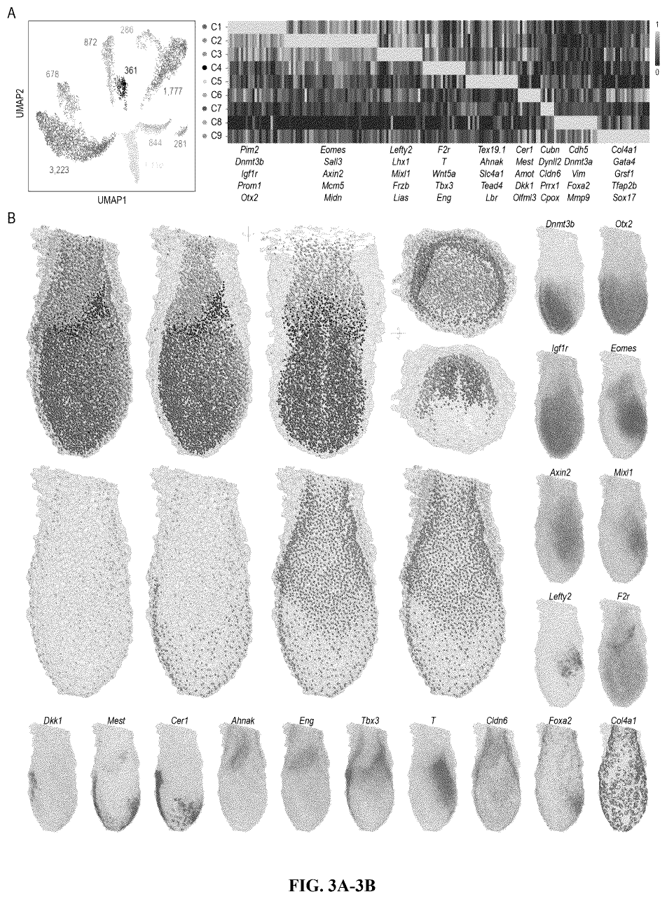

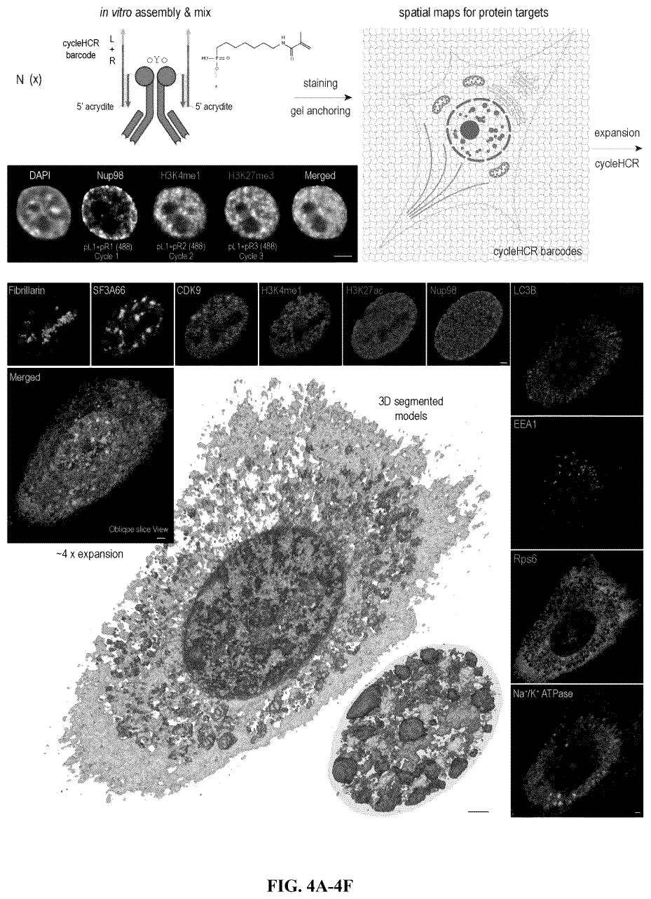

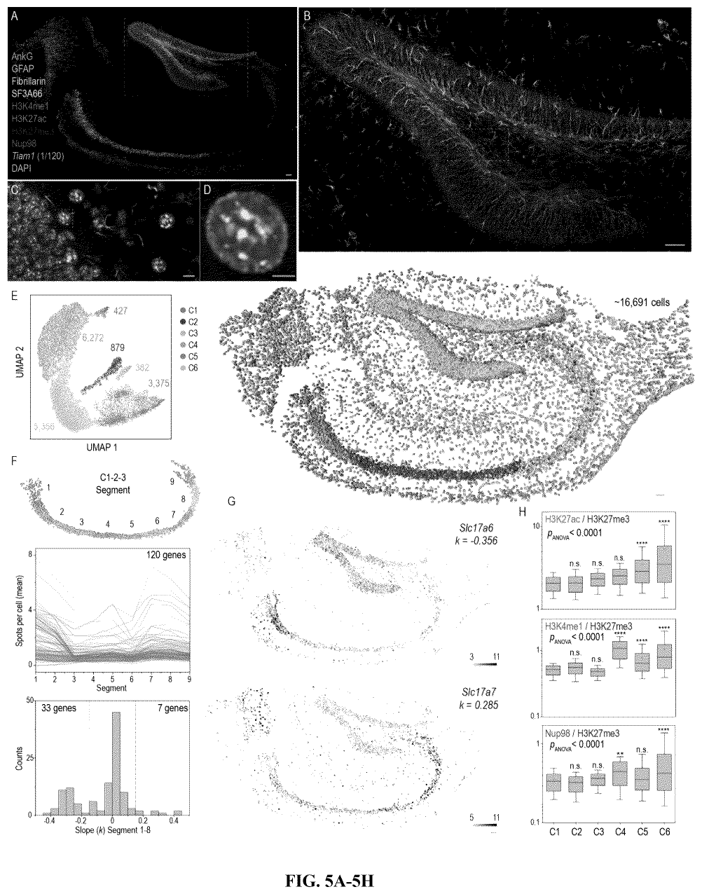

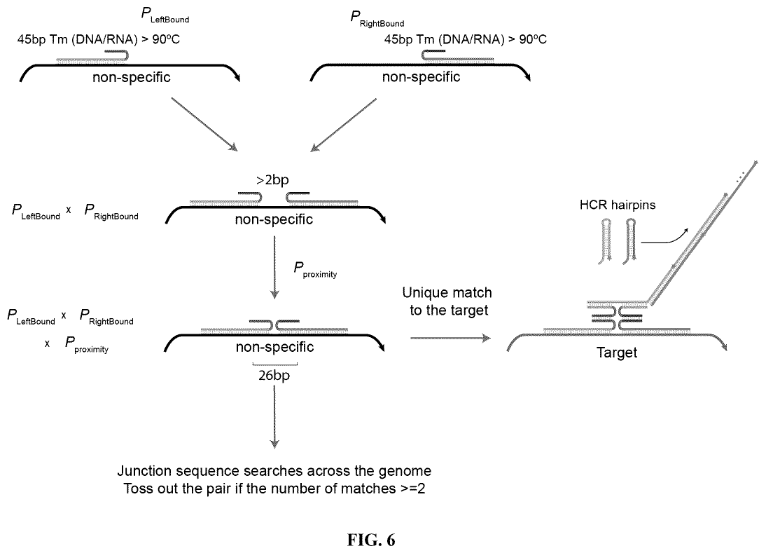

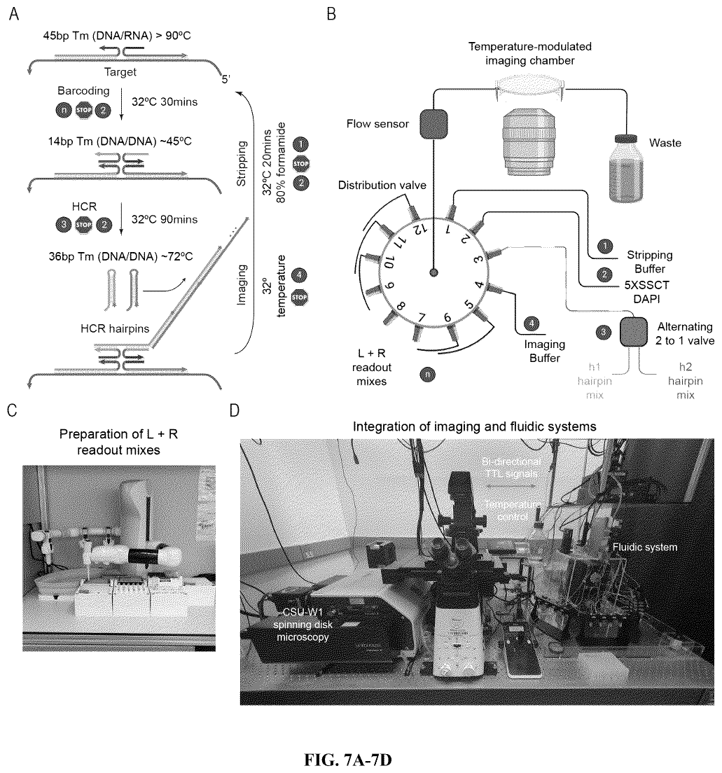

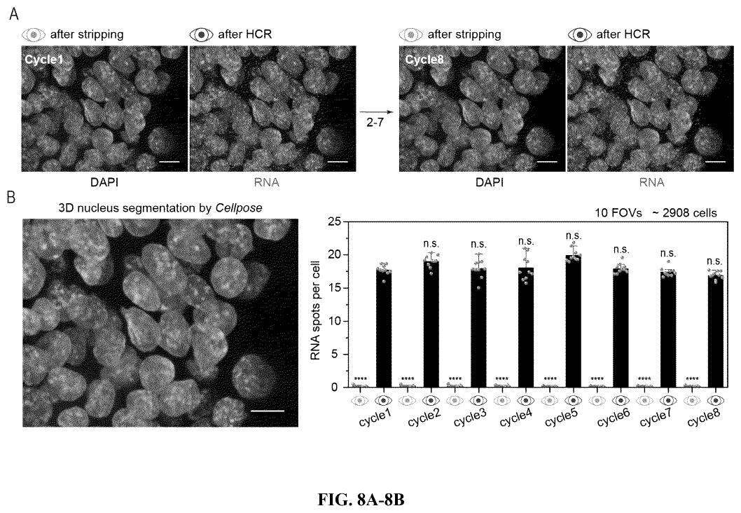

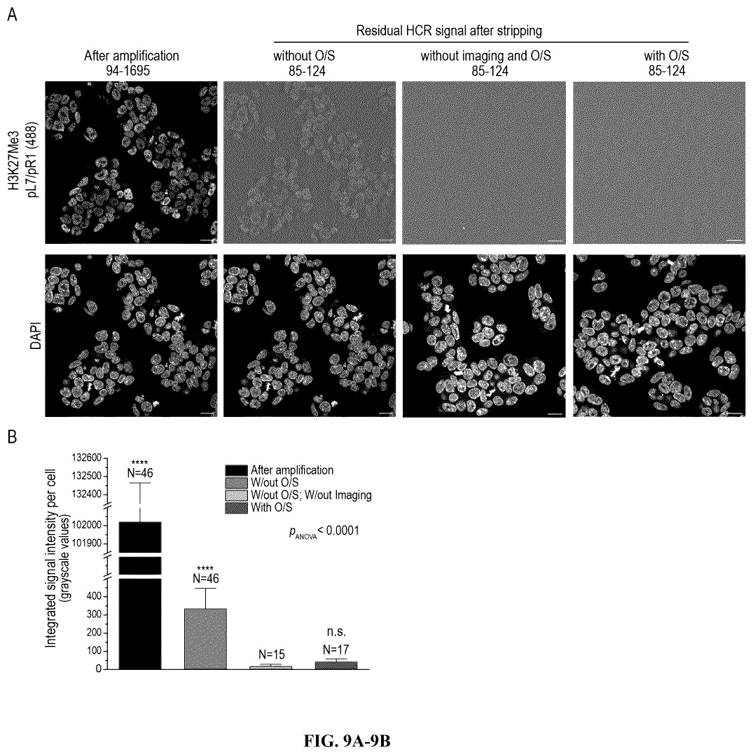

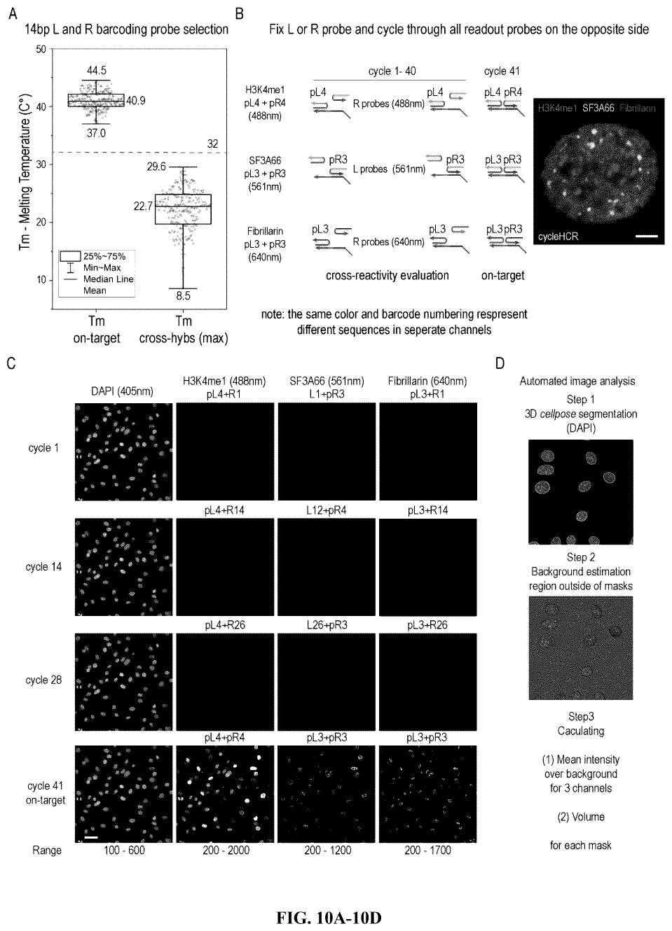

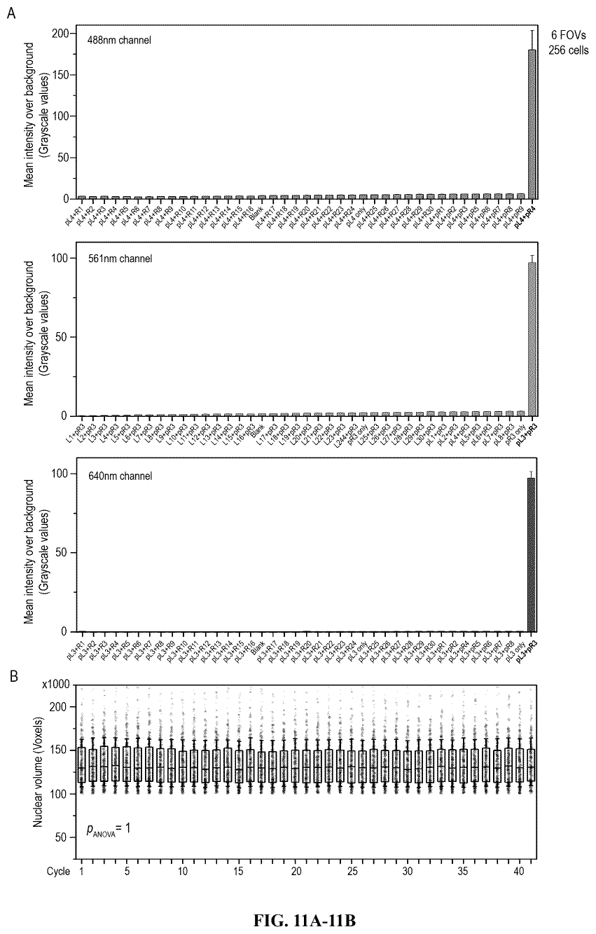

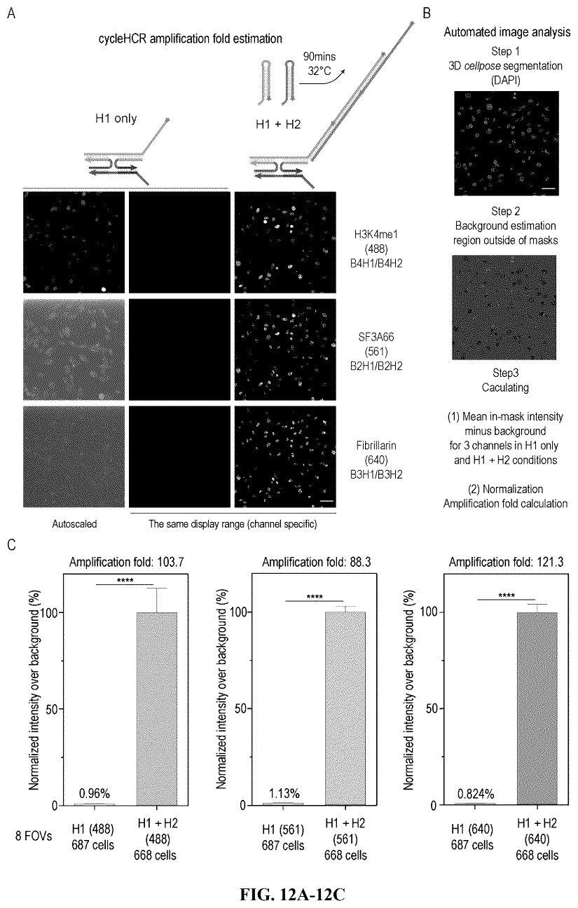

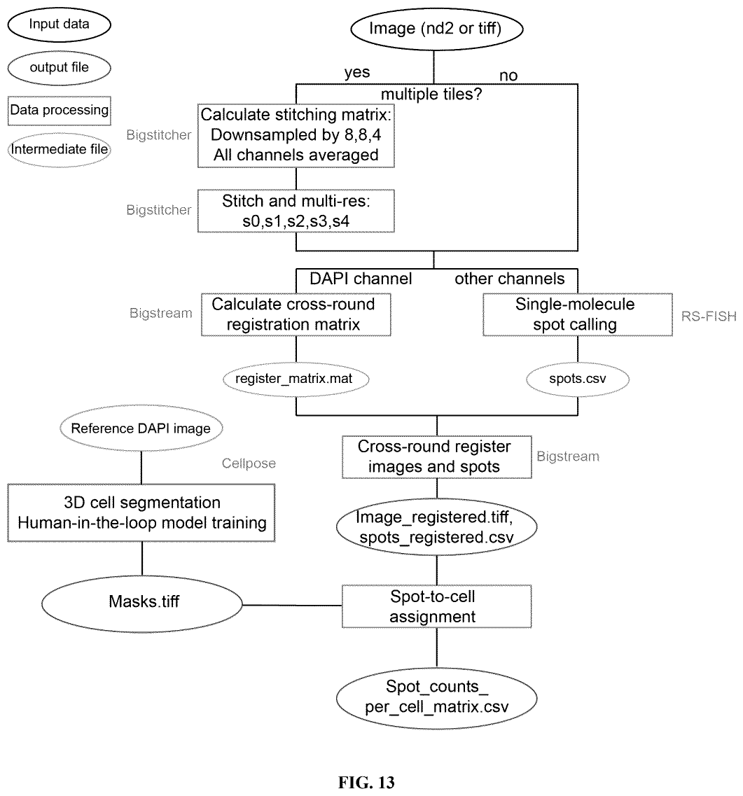

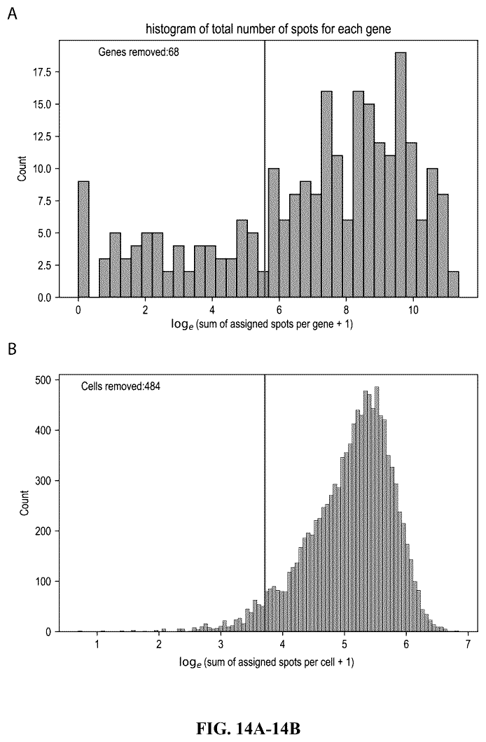

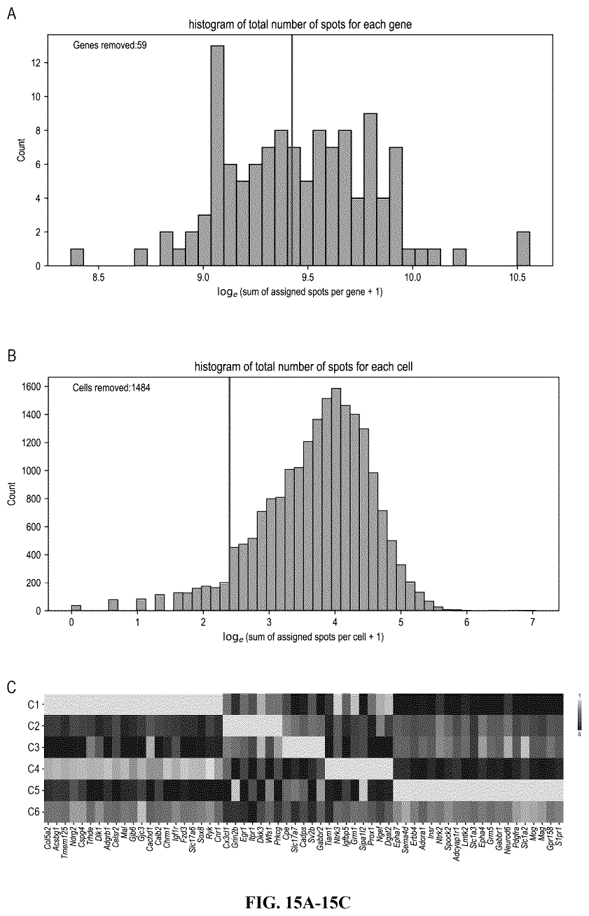

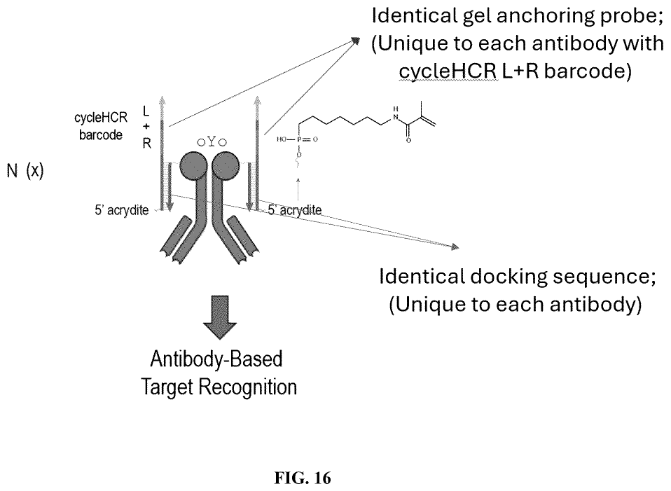

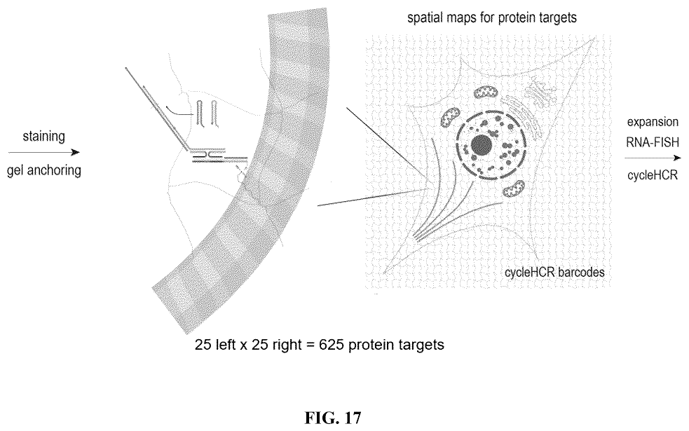

Methods and compositions for spatially mapping a target RNA in a biological specimen are described in this document. As shown in FIG. 1A, a biological specimen can be contacted first with a pair of primary probes followed by a pair of readout probes, and then with a first and a second HCR amplifier sequence.

As described herein, the pair of primary probes includes a left primary probe having a forward PCR sequence, a left target complementary sequence, a left barcode sequence, and a reverse PCR sequence and a right primary probe having the same forward PCR sequence that is present on the left primary probe, a right barcode sequence, a right target complementary sequence, and the same reverse PCR sequence that is present on the left primary probe.

Also as described herein, the pair of readout probes includes a left readout probe having a left HCR initiation sequence and a sequence that is complementary to the left barcode sequence and a right readout probe having a right HCR initiation sequence and a sequence that is complementary to the right barcode sequence.

The methods described herein can be multiplexed by using a plurality of pairs of primary probes and/or a plurality of pairs of readout probes, and it would be understood that pairs or primary probes and/or pairs of readout probes can be provided in a library representing dozens or hundreds of possible probe pair combinations.

Further as described herein, the first and the second HCR amplifier sequence each forms a hairpin and has a detectable label attached thereto, and a portion of the first HCR amplifier sequence and a portion of the second HCR amplifier sequence are complementary to one another. Detectable labels are known in the art and include, without limitation, a fluorophore or a member of a pair of fluorophores.

At this point, the biological specimen can be exposed to conditions under which hybridization chain reaction (HCR) occurs, and labeled HCR products are produced. HCR enables small components to enter a biological specimen and, at the appropriate time under the appropriate conditions, autonomously grow labeled amplification polymers at the site of target RNA within the specimen. HCR relies on two labeled hairpins that store the energy required to drive a conditional self-assembly cascade upon exposure to a suitable initiator. Probes that bind the target RNA and reconstruct the split initiator sequence described herein triggers a chain polymerization reaction of alternating hairpins, leading to the labeled HCR product bound to the target RNA.

Methods of imaging the labeled HCR products in the biological specimen are known in the art and are dependent upon the particular detectable label(s) used. As described herein, when fluorophores are used, the biological specimens can be evaluated using fluorescent microscopes, fluorometers, spectrofluorometers, and fluorescence plate readers. The images that are generated provide a spatial map of the target RNA in the biological specimen.

One of the benefits of the methods and compositions described herein is that the HCR products and the readout probes can be stripped from the biological specimen and the biological specimen can be probed with a different pair of readout probes. In addition, the methods and compositions described herein can be used to evaluate the quality of the RNA in the biological specimen.

The methods described herein readily can be made high-throughput via the use of automated techniques and robotic equipment.

### Spatial Mapping of One or More Target Proteins

Methods and compositions for spatially mapping a target protein in a biological specimen also are described in this document using an antibody complex that binds specifically to the target protein. As shown in FIG. 4A, an antibody complex includes two docking sequences having identical or essentially identical sequences covalently attached to the antibody, and two gel anchoring probes having identical or essentially identical sequences that are hybridized to the two docking sequences. FIG. 4A depicts the two same-sequence docking sequences attached to the antibody via a linker (e.g., a light-activated oYo linker), and shows that each of the gel anchoring probes includes a left barcode sequence, a right barcode sequence, and at least one 5′ acrydite modifications. As used herein, essentially identical can refer to sequences having, e.g., at least 95%, 96%, 97%, 98%, or 99% sequence identity to one another or sequences having less than 10 nucleotide differences (e.g., less than 5, 4, 3, 2, or 1 nucleotide differences).

After the antibody complex is produced and introduced into the biological specimen, the biological specimen can be immobilized in a gel via the one or more 5′ acrydite modifications on the gel anchoring probes.

A pair of readout probes (i.e., having a left readout probe and a right readout probe) is introduced first into the gel-immobilized biological specimen, following by the introduction of a first and second HCR amplifier sequence.

As described herein, the left readout probe includes a left HCR initiation sequence and a sequence that is complementary to the left barcode sequence, while the right readout probe includes a right HCR initiation sequence and a sequence that is complementary to the right barcode sequence.

Further as described herein, the first and the second HCR amplifier sequence each forms a hairpin and has a detectable label attached thereto, and a portion of the first HCR amplifier sequence and a portion of the second HCR amplifier sequence are complementary to one another. Detectable labels are known in the art and include, without limitation, a fluorophore or a member of a pair of fluorophores.

At this point, the biological specimen can be exposed to conditions under which hybridization chain reaction (HCR) occurs, and labeled HCR products are produced. As discussed herein, HCR enables small components to enter a biological specimen and, at the appropriate time under the appropriate conditions, autonomously grow labeled amplification polymers at the site of target protein within the specimen. HCR relies on two labeled hairpins that store the energy required to drive a conditional self-assembly cascade upon exposure to a suitable initiator. Probes that bind the target protein and reconstruct the split initiator sequence described herein triggers a chain polymerization reaction of alternating hairpins, leading to the labeled HCR product bound to the target protein.

Methods of imaging the labeled HCR products in the biological specimen are known in the art and are dependent upon the particular detectable label(s) used. As described herein, when fluorophores are used, the biological specimens can be evaluated using fluorescent microscopes, fluorometers, spectrofluorometers, and fluorescence plate readers. The images that are generated provide a spatial map of the target RNA in the biological specimen.

The methods described herein readily can be made high-throughput via the use of automated techniques and robotic equipment.

One of the benefits of the methods and compositions described herein is that the HCR products and the readout probes can be stripped from the biological specimen and the biological specimen can be probed with a different pair of readout probes.

### Nucleic Acids and Polypeptides

As used herein, nucleic acids can include DNA and RNA, and includes nucleic acids that contain one or more nucleotide analogs or backbone modifications. A nucleic acid can be single stranded or double stranded, which usually depends upon its intended use.

Nucleic acids and polypeptides can differ in sequence from, for example, a reference sequence, e.g., nucleic acids and polypeptides can have at least 80% sequence identity (e.g., at least 85%, 90%, 95%, or 99% sequence identity) to a nucleic acid or polypeptide reference sequence. In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

A skilled artisan will appreciate that changes can be introduced into a nucleic acid molecule, thereby leading to changes in the amino acid sequence of the encoded polypeptide. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5 (Suppl. 3):345-352), which provides frequency tables for amino acid substitutions), and a non-conservative substitution is one in which an amino acid residue is replaced with an amino acid residue that does not have a similar side chain.

Fragments also are included in the disclosure. Suitable nucleic acid fragments typically are those fragments that encode a polypeptide having functional activity. These fragments can be referred to as “functional fragments,” although it is understood that it is not the nucleic acid that possesses functionality.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A vector (or construct) containing a nucleic acid (e.g., a nucleic acid that encodes a polypeptide) also is provided. Vectors, including expression vectors, are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST)).

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Vectors as described herein can be introduced into a host cell. As used herein, “host cell” refers to the particular cell into which the nucleic acid is introduced and also includes the progeny or potential progeny of such a cell. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, CA).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

### Articles of Manufacture

One or more of the compositions necessary to practice the methods described herein can be provided in an article of manufacture.

In one instance, an article of manufacture for spatially detecting target RNA can include a plurality of pairs of primary probes and a plurality of pairs of readout probes as described herein. An article of manufacture also can include a first and a second HCR amplifier sequence as described herein.

In another instance, an article of manufacture for spatially detecting target protein can include a pair of docking sequences and a pair of gel anchoring probes as described herein. An article of manufacture also can include a pair of readout probes and/or a first and a second HCR amplifier sequence as described herein. An article of manufacture for spatially detecting target protein also can include one or more proteins or affinity reagents (e.g., antibodies, DARPins, nanobodies, other synthetic binders) that bind one or more targets in the biological specimen, and further can include a linker (e.g., a light-activated oYo linker). An article of manufacture for spatially detecting target protein additionally can include the reagents required for one or more 5′ acrydite modifications.

It would be appreciated that any of the articles of manufacture described herein also can include one or more detectable labels (e.g., fluorophores or pairs of fluorophores), any of the reagents necessary for hybridization chain reaction (HCR) to occur, one or more reagents necessary for fixing the biological specimen, and or one or more reagents necessary for stripping HCR products and probes from the biological specimen.

Articles of manufacture provided herein also can contain a package insert or package label having instructions thereon for using the various components (e.g., primary probes, docking sequences, gel anchoring probes, readout probes, HCR amplifier sequences, detectable labels, and/or linkers) to spatially map a target RNA or protein in a biological specimen. Articles of manufacture may additionally include reagents for carrying out the methods disclosed herein, where reagents can include, without limitation, buffers, enzymes, co-factors, and combinations thereof.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Cell Lines

Mouse ESCs were maintained in Knockout DMEM (GIBCO 10829-018) supplemented with 10% fetal bovine serum ES cell qualified (ATCC® SCRR-30-2020™), 1× GlutaMAX™ Supplement 100× (GIBCO, 35050-061), 1× MEM non-essential amino acids solution, 100× (GIBCO, 11140050), 1× Antibiotic-Antimycotic (GIBCO, #15240062), 0.1 mM 2-mercaptoethanol (GIBCO, 21985-023), 1000 U/mL LIF (EMD Millipore, ESG1106), 1 μM PD03259010 (Millipore Sigma, PZ0162), 3 μM CHIR99021 (StemCell Technologies, 72052). Primary mouse embryonic fibroblasts (pMEFs) were maintained in low glucose DMEM (GIBCO, 12320032) supplemented with 10% fetal bovine serum, 1× GlutaMAX™ Supplement and 1× Antibiotic-Antimycotic. Cell lines were maintained at 5% CO2 at 37° C.

### Example 2

### Mice and Tissue Sections

C57BL/6J mice (3 months old) were used in our study. Primary rodent work was performed in accordance with protocols approved by the Janelia Research Campus Institutional Animal Care and Use Committee (IACUC) guidelines. Mice were housed in a 12 h light/dark cycle. Animals were anesthetized with isoflurane and perfused with RNase-free PBS (15 ml) followed by 50 mL 4% paraformaldehyde (PFA) buffered to 0.1 M Phosphate Buffer (pH 7.4). After dissection, brains were fixed in 4% PFA overnight, rinsed into 1×PBS, immersed in 30% (w/v) RNase-free Sucrose (Sigma-Aldrich, S7903) in 1×PBS to shake overnight, until the brain sank. Cerebellum was dissected out and embedded in OCT (Fisher Scientific, 23-730-571). 200 mm cryosections of the cerebellum were cut around Bregma-5.80 mm. Cryosections were directly mounted onto PDL-coated silanized 40 mm coverslips.

For flash frozen tissue, animals were anesthetized with isoflurane, checked for toe-pinch pain response, and decapitated before the hippocampus was dissected out. The hippocampus was oriented in OCT within a Peel-A-Way™ embedding molds (Millipore Sigma, E6032), and snap frozen by placing the mold into a dry ice/ethanol mixture (an approximate ratio of 5:1) until the OCT had frozen solid (about 5 min). OCT blocks were acclimated to −14° C. in Leica CM 3050S Cryostat for 1 hour before 40 mm cryosections were collected coronally around Bregma −1.46 mm. Cryosections were directly mounted onto PDL-coated silanized 40 mm coverslips.

Mouse embryos were obtained from non-hormone primed Hsd: ICR (CD-1) females (Envigo) mated in house with B6D2F1 (C57BL/6×DBA) males. Insemination was verified the next morning by the presence of a copulatory plug, and this day was defined as E0.5 days post fertilization (d.p.f). Timed pregnant females were euthanized by cervical dislocation, and the embryos were recovered in 4% PFA (Electron microscopy sciences) diluted in RNase-free PBS at 4° C. Embryos were removed from the uterus and dissected from the decidua and parietal yolk sac using fine forceps, leaving the ectoplacental cone attached to the egg cylinder. Subsequently, the embryos were fixed in fresh 4% PFA (RNase-free) overnight at 4° C., washed twice for 5 minutes with RNase-free PBS-T (1% PBS; Tween 20) on ice, and dehydrated in a series of methanol/PBS-T dilutions (10 minutes each) on ice as follows: 1) 25% MeOH/75% PBS-T; 2) 50% MeOH/50% PBS-T; 3) 75% MeOH/25% PBS-T; 4) 100% MeOH. Lastly, embryos were incubated at −20° C. overnight (>16 hours).

### Example 3

### Coverslip Cleaning and Coating

40 mm coverslips (Bioptechs, 40-1313-0319) were cleaned with the following modifications. For the silanized coating, coverslips were immersed in a solution of 0.2% Triethylamine (Sigma Aldrich, 471283) and 0.3% allyltrichlorosilane (Sigma Aldrich, 107778) in Chloroform for 30 minutes. For tissue adhesion, silanized coverslips were coated with 100 mg/ml poly-D-lysine (Sigma P7280) dissolved in 1×PBS overnight. For cell adhesion, silanized coverslips were coated with 1 mg/ml Laminin (Gibco, A29248) for 2 hours at 37° C.

### Example 4

### cycleHCR Probe Design and Assembly

cycleHCR primary probe selection

For precise single-shot RNA imaging using cycleHCR, a comprehensive multi-step probe selection process is applied to each target transcript, ensuring high specificity and robust signal amplification. This process encompasses: 1) A sliding window approach is employed to identify an optimal 92 bp sequence within the target transcript for probe design. This sequence is divided into two segments of 45 bp each for the left and right probe pairs, with a 2 bp gap between them. The selection criteria for these probe halves include: a maximum secondary structure melting temperature (Tm) of less than 76° C.; a maximum cross-hybridization Tm of less than 72° C.; a GC content ranging from 30% to 90%; exclusion of sequences with six consecutive identical nucleotides (GGGGGG (SEQ ID NO:5), CCCCCC (SEQ ID NO:6), TTTTTT (SEQ ID NO:7), AAAAAA (SEQ ID NO:8)) and a minimal gap of 2 bp between any two consecutive probes along the 92 bp region. 2) The DNA-RNA probe binding Tm is calculated for each probe half, ensuring a Tm above 90° C. Such high Tm values are crucial for maintaining the stability of probe-target interactions, particularly under stringent stripping conditions. 3) The final step in probe selection involves a thorough specificity check against the entire genome. This involves screening for 26 bp junction sequences, representing the unique overlapping region between left and right probe halves. Any probe pair matching more than once in the genome is discarded to eliminate the risk of non-specific targeting.

### L and R Barcoding Probe Sequence Selection

For the selection and assembly of L+R barcoding probes, 14 bp RNA and protein left and right readout probes was initially generated by using the randseq( ) function in Matlab 2022. The probe sequences underwent rigorous screening to adhere to specific criteria, aiming to enhance their specificity and minimize non-specific interactions. These criteria included: 1) Exclusion of sequences containing runs of four or more identical nucleotides (4 or more Gs, Cs, Ts, or As) to prevent formation of secondary structures that could impair hybridization efficiency. 2) Elimination of sequences with the potential to form dimers or hairpin structures, which could interfere with probe-target interactions. 3) Ensuring a GC content between 30% and 60%, balancing hybridization strength and specificity. 4) A melting temperature constraint of less than 45° C. to facilitate selective binding under the assay conditions. 5) Screening each new sequence against all existing sequences in the library to ensure a maximum cross-hybridization melting temperature (Tm) at least 10° C. lower than its own Tm, thereby reducing the likelihood of non-specific binding among probes. Through this method, a total of 230 probes were successfully generated, with 180 designated for barcoding cycleHCR RNA targets and the remaining 50 for barcoding cycleHCR protein targets.

### L and R Barcoding Probe Assembly

The assembly of L and R barcoding probes for cycleHCR involves a division and pairing process to enable specific and efficient Hybridization Chain Reaction (HCR) for each color channel. The process is outlined as follows: 1) The 180 RNA barcoding probes are divided into two sets of 30 Left (L) and 30 Right (R) probes for each color channel. This division ensures a wide range of unique barcode combinations, facilitating the multiplexing capability of the cycleHCR system. 2) To prepare the L and R probes for HCR initiation, we link a specific 18bp split HCR initiator sequence to the 5′ end of each left barcode, separated by a short spacer (AA). Similarly, the right barcode is linked to the 5′ end of the other 18 bp split HCR initiator separated by a short spacer (AA), specific to the same color channel. This initiator sequence was designed to trigger the HCR reaction in a color channel specific manner. 2) Following the assembly, the L and R barcoding probes, now equipped with their respective HCR initiator sequences, are synthesized using Integrated DNA Technology.

### Primary Probe Library Assembly

For each RNA target, we chose between 10 to 25 probe pairs. The construction of these probe pairs involves the assembly of left and right primary probes as follows: 1) Left Primary Probe Assembly: A forward PCR sequence containing a T7 promoter (TAA TAC GAC TCA CTA TAG CGT CAT C) initiates the assembly. This is followed by the first 45 bp sequence of the selected 92 bp segment. A spacer sequence ‘TT’ is inserted next. The 14 bp left barcode sequence comes after the spacer. The assembly concludes with a reverse oligo sequence (CGA CAC CGA ACG TGC GAC AA). 2) Right Primary Probe Assembly: Similar to the left probe, it starts with the same forward PCR sequence with the T7 promoter. The 14 bp right barcode sequence is placed immediately after the promoter. A spacer ‘TT’ follows the barcode. The last 45 bp of the 92 bp primary probe sequence is then added. The assembly is completed with the same reverse oligo sequence (CGA CAC CGA ACG TGC GAC AA). Each assembled probe, whether left or right, ultimately spans a total length of 92 bp.

### Example 5

### DNA Synthesis and Purification

ssDNA libraries were synthesized by Twist Bioscience. dsDNA was amplified using KAPA HiFi HotStart Polymerase (Roche, KK2502) using the following two primer sequences (T7 Forward: 5′-TAA TAC GAC TCA CTA TAG CGT CAT C-3′ (SEQ ID NO: 1); Reverse: 5′-TTG TCG CAC GTT CGG TGT CG-3′ (SEQ ID NO:2)) and purified with DNA Clean and Concentrator-5 Kit (Zymo Research, 11-302). dsDNA was converted to RNA with HiScribe™ T7 High Yield RNA Synthesis Kit (NEB, E2040S) and purified using Monarch® RNA Cleanup Kit (NEB, T2040). An RNA/DNA mix was obtained by retrotranscribing 200 pmol of RNA +200 pmol of the Reserve primer (5′-TTG TCG CAC GTT CGG TGT CG-3′ (SEQ ID NO:3)) with Maxima H Minus Reverse Transcriptase (Thermo Scientific, EP0753) as per manufacturer instructions. The RNA was digested with Thermolabile USER (Uracil-Specific Excision Reagent) II (NEB, M5508) overnight before alkaline hydrolysis in 1M NaOH at 65° C. for 15 minutes. Hydrolysis was neutralized with 1M acetic acid before purifying the libraries using ssDNA/RNA Clean & Concentrator™ (Zymo Research, 50-444-498).

### Example 6

### RNA cycleHCR in Tissues and Cells

### Fixation and Permeabilization

Fixed tissues were washed 3 times in 1×PBS (for 5 minutes each, then incubated in ice-cold methanol (Fisher Chemical., A454-1), on ice, for 20 minutes, 5 minutes in ice-cold [50% methanol, 50% 2×SSC−0.1% Tween] and 5 minutes in cold—[25% Methanol, 75% 2×SSC-0.1% Tween]. 25% methanol was then removed by rinsing three times with 1×PBS and washing once in 1×PBS for 5 minutes (tissue≤40 μm) or 10 minutes (tissue≥100 μm). Tissue was permeabilized in [0.5% Triton, 1×PBS] for 30 minutes at room temperature, followed by 3 washes in 1×PBS for 5 minutes each.

Cells were fixed in 4% paraformaldehyde (PFA) in 1×PBS (Invitrogen, AM9625) for 10 minutes and permeabilized in 0.5% Triton for 15 minutes.

### Gel Embedding and Proteinase K Digestion

After permeabilization, samples were incubated in 0.1 mg/ml Acryloyl-X SE (AcX) for 1 hour at room temperature. AcX (Invitrogen, A20770) was prepared as described (37). Samples were then washed twice in 1×PBS, 10 minutes each time. Before proceeding with the gelation step, samples were incubated for 5 minutes (tissue≤40 mm) or 10 minutes (tissue≥100 mm) in polyacrylamide (PA) solution [4% acrylamide/bis acrylamide (BioRad., 1610154), 60 mM Tris HCl pH 8 (Corning, 460131), 0.3 M NaCl (Corning, 46032]. For gelation, APS (Sigma, A3678) and TEMED (Sigma, T7024) were added to the PA solution with a final concentration of 0.03% and 0.15% respectively. Gelation chamber was assembled. Briefly, a pre-cleaned slide 75×50 mm, thickness 0.96 to 1.6 mm (Corning 2947-75X50) was coated with Gel Slick (Lonza, 182369) for 15 minutes and let air dry for at least 30 minutes. 100 mL of PA solution with APS and TEMED were added to the dry-glass plate and sample was slowly overlaid to avoid air bubbles. After 1.5 hour at room temperature, the gel fully polymerized and sample was gently detached from the glass plate. The silanized coating allows covalent binding of the hydrogel to the 40 mm coverslips, while the gel slick coating prevents the gel from sticking to the glass plate during the polymerization. As a result, when the 40 mm coverslip is gently detached from the glass plate, the gel-embedded sample will remain stably bound to the coverslips during further processing.

Tissue samples were then digested with Proteinase K (NEB, P8107S). Tissue was incubated in [2×SSC, 2% SDS, 0.5% Triton, 1:100 dilution of Proteinase K (800 units/ml; NEB P8107S)] for 16 hours in a humid chamber at 37° C. Cells were digested.

RNA Quality Control and Hybridization with ssDNA Libraries

Steps that required small volumes (100-200 mL) were performed in custom-made hybridization chambers of the following dimensions: [0.12 mm chamber depth/20 mm diameter] for samples≤40 μm; [0.5 mm chamber depth/19 mm diameter] for tissue 100-200μm; [1 mm chamber depth/10 mm diameter] for embryos. To assemble the hybridization chambers, a 13-in-1 heavy duty hollow punch sets was used to cut the internal diameters indicated above from a round-silicon sheet (BIOPTECHS, 1907-1422-500 and 1907-1422-500). The prepared gasket was then securely affixed to a pre-cleaned slide 75×50 mm, thickness 0.96 to 1.6 mm (Corning 2947-75X50) using 100% silicone sealant (GORILLA). For the 0.12 mm×20 mm chamber, am imaging spacers (Grace Bio-Labs Cat. #654006) was attached to the pre-cleaned slide as described above.

Before incubating the sample with primary probes, RNA quality was assessed using our Alexa Fluor 488-conjugated-readout probe (RO22: 5′-GCC AAG ATG GAG TTA-3′ (SEQ ID NO: 4)) targeting ribosomal RNAs. After proteinase K digestion, samples were washed twice in 5×SSC-T [5×SSC, 0.1% Tween] for 15 minutes. Ribosome probes were diluted in 10% EC Buffer [10% ethylene carbonate (Sigma, E26258), 10% dextran sulfate (Sigma, D4911), 2×SSC) at the following concentrations: 100 nM RO22 probes (samples≤40 μm) or 200 nM (tissue ≥ 100 mm). Samples were incubated in the appropriate concentration of RO22 for 1 hour at room temperature and washed once (samples≤40 μm) or twice (tissue≥100 mm) in 10% formamide solution [10% deionized formamide (Ambion, AM9342), 0.1% triton, 2×SSC) for 10 minutes. Before imaging, samples were then washed in 5×SSC-T before staining nuclei with 5 mg/ml DAPI.

After assessing RNA quality, samples were incubated with primary probe libraries. Primary probes were added to a total volume of 100 uL (for 0.12 mm×20 mm or 1 mm×10 mm diameter chambers) or 250 μL (for 0.5 mm/19 mm chamber) hybridization solution [50% deionized formamide, 10% Dextran Sulfate, 2×SCC] in the following amount: ˜2ug of ssDNA libraries targeting 10 RNA species or less, ˜25 μg of ssDNA libraries targeting RNA transcripts from 120 genes, ˜50 μg of ssDNA libraries targeting RNA transcripts from 279 genes. Samples were hybridized with primary probes at 37° C. for about 20 hours in RapidFISH Slide Hybridizer Oven (Boekel Scientific, Cat. #13-245-230). High stringent wash was carried out in 80% formamide solution [80% formamide, 4×SSC, 0.1% Triton) at 32° C. for 20 minutes before readout probe hybridization.

### Example 7

### Readout Probe Hybridization and Hybridization Chain Reaction (HCR)

Samples were incubated with 200nM readout probes in 10% EC Buffer for an hour at room temperature, rinsed three times in 5×SSC-T and subsequently washed once (samples≤40 μm) or twice (tissue≥100 mm) in 10% formamide solution [10% deionized formamide, 0.1% triton, 2×SSC) for 10 minutes. Samples were then washed three times in 5×SSC-T before HCR. Expanded samples were incubated with 200 nM readout probes for 3 hours and washes performed as described above.

For HCR, H1 and H2 amplifiers (HCRTM Amplifiers: B4 fluorophore 488, B3 fluorophore 647, B2 fluorophore 560, Molecular Instruments, Inc.) were activated according to the manufacturer's instructions. After activation, H1 and H2 were added to the Amplification buffer [10% dextran sulfate, 0.1% Tween, 5×SSC) in a 1:100 ratio. In non-expanded samples, HCR was carried out for 1.5 hour at 32° C. and washed twice in 5×SSC-T for 10 minutes before imaging. In expanded samples, HCR was carried out for 3 hours at 32° C.

### Example 8

### Stripping and Reprobing

Readout probes and HCR chains were removed by incubating the sample in 80% formamide solution (80% deionized formamide, 0.1% triton, 4×SSC) for 20 minutes at 32° C. Samples were then rinsed three times in 5×SSC-T and washed once in 5×SSC-T for 10minutes before reprobing with readout probes for the consecutive round.

### Example 9

### Sample Preparation for cycleHCR Protein Imaging

### Fixation and Permeabilization

40 mm cryosections were removed from −80° C. and immediately fixed. Tissues and cells were both fixed in 4% PFA for 10 minutes and permeabilized in 0.5% Triton in 1×PBS for 15 minutes at room temperature. After permeabilization, samples were incubated for 1hour in blocking solution [0.25% Triton, 0.5 mg/ml salmon sperm DNA (Fisher Scientific AM9680), 10 mg/ml nucleases-and proteinases-free BSA (Sigma, 126609), 1×PBS]. During blocking, the antibodies conjugated with a unique docking oligo were assembled with the acrydite™M 5′-gel docking oligos (IDT).

In Vitro Assembly of Antibodies-oYo-oligos with Acrydite™ 5′-Gel Docking Oligos

oYo Link® conjugated with each unique docking oligos were purchased from AlphaThera and crosslinked to the antibodies of interest according to the manufacturer's instructions. For in vitro assembly of gel docking complex, a mix of 1 ml oYo-crosslinked antibodies, 7 mM acrydite™ 5′-gel docking oligos and 10% dextran sulfate in 1×PBS was incubated for 1 hour at room temperature on an orbital shaker at 100 rpm. Antibody staining

Pre-assembled gel docking complexes were combined in 100 ml blocking solution and incubated with the sample overnight in a humid chamber at 4° C. To preserve RNA during incubation with primary antibodies, 6 units of SUPERase·In™ RNase Inhibitor (Invitrogen, AM2694) were added to the antibody mix. The next day, samples were washed three times in 1×PBS, before post-fixation in 4% PFA for 10 minutes. Samples were washed in 1×PBS and then incubated with 0.1 mg/ml Acryloyl-X SE (AcX) for 1 hour at room temperature. After Acx modification, non-expanded samples were embedded in a thin-hydrogel layer, digested with proteinase K and incubate with readout probes as described in RNA FISH section. For simultaneous detection of protein and RNA, samples were incubated with primary probes after proteinase K digestion as described in RNA FISH section.

### Example 10

### Protein cycleHCR with Expansion

Protein cycleHCR was combined with expansion microscopy. Samples were prepared as described in Protein cycleHCR. After overnight incubation with the mixture containing the pre-assembled gel docking complexes, samples were treated with 200 μg/mL acryloyl-X SE in 1×PBS for 1 hour at room temperature and rinsed twice with 1×PBS for 15 minutes each. Samples were then embedded in TREx1000 gelation solution [TREx1000 1 M sodium acrylate (Sigma, 408220), 14% acrylamide (Bio-Rad, 1610140), 1000 ppm N,N′-methylenebisacrylamide (bis, Sigma, M7279), 1XPBS, 2000 ppm APS (Sigma, A3678), 2000 ppm TEMED (Sigma, T7024), and 100 ppm 4-hydroxy TEMPO (4HT, Sigma, 176141)] on a chamber composed of a Gel Slick-treated glass slide and 1-layer Scotch Magic

Tape spacers (˜56 μm, 3M, #810) (see details in (39)). Gel was polymerized for 3 hours at room temperature to prevent denaturing the gel docking strand from the antibody docking strand. The cell-embedding gel was then detached from the coverslip, cut into smaller pieces, and digested in proteinase K (NEB, P8107S) diluted 1:100 in proteinase K digestion buffer (50 mM Tris-HCl pH 8, 500 mM NaCl, 1 mM EDTA, 0.5% Triton X-100, and 1% SDS) overnight at 37° C. The gel was washed 3 times in 1×PBS and three times in nuclease-free water for 30 minutes each time to fully expand. The expanded gel was then incubated in re-embedding gelation solution for 30 minutes on ice. The re-embedding gelation solution contains 44% acrylamide, 0.1% N,N′-methylenebisacrylamide (bis), 0.2% TEMED, 0.01% 4HT, and 0.2% APS. The re-embedding gel was then allowed to polymerize on a cleaned, silanized, and poly-D-lysine coated 40 mm coverslip in a chamber with 4-layer-Scotch Magic Tape spacers for 1 hour at 37° C. After re-embedding, the samples were loaded on our automated microfluidics imaging system to acquire protein cycleHCR data.

### Example 11

### Antibodies

The following rabbit antibodies were used at 1:100 dilution unless otherwise indicated. Anti-Fibrillarin (Abcam, ab5821), Anti-Histone H3 (mono methyl K4) (Abcam, ab8895), Anti-Histone H3 (acetyl K27) (Abcam, ab177178), anti-Tri-Methyl-Histone H3 (Lys27) (Cell Signaling, 9733), anti-GFAP (Abcam, ab278054) used at 1:200, anti-Iba1 (Abcam, ab178846), anti-Nup98 (Cell Signaling, 2598) used at 1:30 dilution, anti-Tomm20 (Abcam, ab186735), anti-Calnexin (Abcam, ab22595), anti-RPS6 (Abcam, ab225676), anti-RAB7 (Abcam, ab126712), anti-EEA1 (Abcam, ab2900), anti-LAMP2A (Abcam, ab18528), anti-LC3B (Abcam, ab192890), anti-Na/K ATPase (Abcam, ab76020), anti-Fibrillin (Abcam, ab53076), anti-CDK9 (Abcam, ab239364).

The following mouse antibodies were used at 1:100 dilution: anti-SF3a66 (Abcam, ab77800), anti-SC35 (Abcam, ab11826), anti-Ankyrin-G (Antibodies Inc., 75-146). anti-Syntaxin 6 (BD Biosciences, 610636), anti-Lamin B1 (Abcam, ab8982).

### Example 12

### cycleHCR Fluidics and Imaging System

The fluidics system is designed for precision in flow control and mixing, essential for cycleHCR imaging processes. It incorporates an OB1 flow controller for managing the flow rate through the system, two MUX distribution valves for directing flow to specific channels and a BFS Coriolis flow sensor to provide feedback for accurate flow control. This setup accommodates 10 probe tubes for L+R readout probes, 4 solution types including buffers and washing solutions and 2 hairpin mix tubes—One for H1 hairpin mix and another for H2 hairpin mix. The real-time mixing of H1 and H2 hairpin mixes is regulated by alternating the flow between them using a 2 to 1 MUX valve, ensuring efficient HCR reaction initiation. Flow Rate: Optimized at 150 μl/min to balance efficiency and sample integrity. After primary probe hybridization, samples on a 40 mm coverslip are placed in a closed-top FCS2 chamber for imaging. This chamber is then positioned on a customized stage adapter on the microscope. All buffers used in the following fluidics steps were filtered through 0.22 mm vacuum filter (Corning, 431098).

### Fluidics Steps:

Stripping Buffer (80% formamide, 4×SSC, 0.1% Triton) Incubation (20 mins): Removes bound probes and prepares the sample for the next cycle.

Washing (20 mins): Uses 5×SSC and 0.1% Tween buffer with 5 mg/ml DAPI to clean the sample.

Readout Probe Incubation (30 mins): Applies mixed Left and Right probes for a new cycle of target visualization. 200 nM of each readout probes were added to 1.8 mL of hybridization solution (10% Ethylene Carbonate, 5×SSC, 10% Dextran Sulfate)

Washing (20 mins): Another round with 5×SSC with DAPI.

Hairpin Mix Injection (5 mins): Introduces H1 and H2 mixes for signal amplification. H1 and H2 were added to the amplification buffer (5×SSC, 0.1% Tween, 10% Dextran Sulfate) at 1:50 ratio.

HCR Amplification (90 mins): Enables the fluorescent signal to build.

Washing (10 mins) as in step 4: Cleans excess reagents post-amplification.

Imaging Buffer (50 mM Tris HCl pH8, 2 mM Trolox (Sigma, 238813), 1 mg/ml Glucose Oxidase (Sigma, G2133), 1:100 Catalase (Sigma, C3155), 0.8% D-Glucose, 5×SSC) Injection (20 mins): Prepares the sample with an oxygen scavenger to prevent photo-crosslinking and enhance signal strength.

TTL Signal Activation: Triggers the microscope for imaging after a final 10-minute incubation.

### Microscopy:

The Nikon CSU-W1 spinning disk microscope, equipped with advanced features, is utilized for high-resolution imaging in cycleHCR technology. This setup includes:

A 25× CFI PLAN Apochromat Lambda S silicone oil immersion objective with a numerical aperture (N.A.) of 1.05 and a working distance of 0.55 mm, ideal for imaging intact mouse embryos and protein cycleHCR imaging in expanded primary mouse fibroblasts.

A 40× CFI PLAN Apochromat Lambda S silicone oil immersion objective with an N.A. of 1.25 and a working distance of 0.3 mm, suited for imaging brain tissue slices.

An uniformizer: Ensures even illumination across the field of view.

6 Laser Lines (405 nm, 514 nm, 561 nm, 594 nm, 640 nm): Provide a range of excitation wavelengths for versatile fluorophore excitation.

A Hamamatsu BT Fusion Camera: Captures high-quality images with efficient signal detection.

For the 25× objective, the microscope operated in ultraquiet mode with a fixed framerate of 5.1Hz, optimizing conditions for sensitive samples like intact mouse embryos and expanded primary mouse fibroblasts. For the 40× objective, imaging of brain tissue slices was performed with a 100 ms exposure in the standard camera readout mode. The Nikon's Perfect Focusing System (PFS) maintained the Z position of the objective between imaging rounds, critical for long-term imaging experiments. TTL signals automated the transition between imaging rounds and fluidic cycles, streamlining the cycleHCR process.

Temperature control during imaging was ensured by heating the objective with a Tokai Hit Lens Heater controlled by a TPi controller (TPiE-LH), while the imaging chamber temperature was regulated using a Bioptechs FCS controller.

### Example 13

### Image Stitching

Large images composed of multiple 3D tiles were aligned using BigStitcher and the corresponding BigStitcher-Spark framework for distributed execution (github.com/JaneliaSciComp/BigStitcher-Spark). After re-saving image data into the multi-resolution N5 format, alignment was performed independently for each imaging round, initialized using the 20% overlap (in xy) between neighboring tiles. The calculation of the final 3D affine transformation for each tile consists of several steps: pairwise shift calculation using phase correlation, pre-viewing and filtering pairwise shifts, global optimization of pairwise shifts, and affine refinement of the alignment using the Iterative Closest Point (ICP) algorithm. The pairwise shifts were calculated using 8×8×4 (xyz) downsampling and averaging intensity from all channels, ICP refinement was also computed using 8×8×4 downsampling. The tiles were fused either by weighted average fusion or the one-tile-wins strategy. The weighted average fusion strategy computes the values of pixels in the overlapping regions using a distance-weighted (from the tile boundaries) average, which was used for visualizing protein labeling. The one-tile-wins fusion strategy does not perform averaging in overlapping areas, but instead copies (and interpolates) pixel values from the input tile that was imaged first (out of all overlapping tiles at any given pixel). The one-tile-wins strategy was used for calling single-molecule spots from RNA labeling. This strategy solves decreased spot-detection frequency in the overlapping regions due to averaging pixel values when the same single-molecule spot is slightly misaligned in the different overlapping tiles. The Nextflow pipeline for image processing and stitching is available at github.com/liulabspatial/cycleHCR.

### Example 14

### Cross-Cycle Image Registration

To adjust for shifts in the sample or field of view during multiple cycles of imaging, the images of different rounds were registered to the image from a reference round using the Python package bigstream (version 1.2.9). The reference round was chosen based on manual inspection, and all other rounds were transformed such that the DAPI channels across different rounds align in 3D space.

For single-tile images, the global affine transformation was sufficient. The affine transformation matrix was obtained using the alignment_pipeline function with a downsampling factor of two and a subsampling factor of two. The obtained transformation matrix was then used in the apply_transform function to perform uniform translation, scale, sheer, and rotation on all pixels.

For stitched multi-tile images, the global affine and local deform transformations were sequentially performed. The affine matrix was calculated as above, and then the deform matrix was calculated using the distributed_piecewise_alignment_pipeline function initialized with the affine matrix. The two matrices were provided as sequential steps, affine and then deform, in the distributed_apply_transform function to first perform the global affine transformation to the whole image, followed by local deformable transformation uniquely defined for each pixel. The parameters and codes for bigstream are available at github.com/liulabspatial/cycleHCR.

### Example 15

### Cell Segmentation

For 3D nucleus segmentation of the mouse embryo, a unified custom model was trained using the human-in-the-loop feature in Cellpose 2 on xy and yz slices in which nuclei were manually labeled. Subsequently, 3D segmentation function was utilized to perform the segmentation across all dimensions. Masks less than 1000 voxels were filtered out. In contrast, for 3D nucleus segmentation of the hippocampal slice, two distinct custom models were trained separately for the xy and yz orthogonal views, again utilizing the human-in-the-loop feature in Cellpose 2. A custom 3D segmentation procedure was then implemented by computing the xy flows using the xy model and computing the yz and xz flows using the yz model. Next, the consensus cell flow was calculated by averaging across xy, yz and xz flows and then the dynamics were run on the flows to compute the masks. Masks less than 1000 voxels were filtered out.

The segmentation accuracy was estimated by human inspection of raw DAPI images and corresponding masks. Further manual curation and size filtering to eliminate potential oversized doublets were performed using ORS Dragonfly software.

The parameters and codes for Cellpose segmentation are available at github.com/liulabspatial/cycleHCR/.

### Example 16

### Spot Detection and Spot-to-Cell Assignment

The spot detection process involved three-dimensional single molecule localization using RS-FISH. Specific parameters for each experiment were initially selected using the interactive feature provided by RS-FISH. These parameters were carefully chosen to minimize false positive detections. Importantly, the same parameters were consistently maintained across all images within each series.

Spot-to-cell assignment was achieved by rounding the x, y, z coordinates of each spot. Subsequently, cell assignment was determined by matching the rounded x, y, z voxel value of the labeled cell mask image.

The parameters and codes for RS-FISH localization and spot-to-cell assignment are available at github.com/liulabspatial/cycleHCR/.

### Example 17

### Mask-Based Image Quantification

We utilized the regionprops3( ) function in Matlab 2023b, to compute center-of-mass and intensity measurements on labeled and grayscale image volumes.

### Example 18

### UMAP Analysis

The cell-by-gene matrix was filtered based on the total number of spots per gene and the total number of spots per cell. The total counts per gene were plotted as a histogram on a logarithmic scale. For the embryo data, Otsu's method of thresholding was used to remove genes with low total counts using the Python package scikit-image (version 0.23.1). Cells with less than 40 total counts were removed, resulting in the removal of approximately 5% of cells. For hippocampus data, Li's method of threshold was used to remove genes with low total counts. Cells with less than 10 total counts across the retained genes were removed, resulting in the removal of approximately 7.5% of cells.

The filtered counts matrix was used as input for generating UMAP using the Python package Uniform Manifold Approximation and Projection (UMAP) (version 0.5.5). The cells were clustered in an unsupervised manner using Python package HDBSCAN (version 0.8.33). The parameters and codes used for UMAP and HDBSCAN are available at github.com/liulabspatial/cycleHCR.

### Example 19

### Gene-to-Cluster Assignment

We assigned each gene to one of the identified clusters based on average counts per cell in each cluster. The filtered counts matrix is normalized across each cell by dividing each element of the gene vector by the total counts per cell, thereby normalizing for the potential overrepresentation of certain cells in gene vectors. For each gene, average counts per cell for each cluster are calculated. The gene is assigned to the cluster with the highest average counts. The unnormalized raw counts per cell were plotted in both UMAP and anatomical space to validate gene-to-cluster assignment.

### Example 20

### cycleHCR: Concept and Implementation

Here, we introduce cycleHCR technology that enables high-throughput, single-shot imaging of RNA and protein species within thick tissue specimens. At the core of cycleHCR RNA imaging lies the optimization and selection of 45bp split primary probes (FIG. 1A and FIG. 6) Distinguished by their high melting temperatures (>90° C.) for probe-RNA interactions, these probes ensure robust and stable binding under stringent conditions efficiently clearing away other components such as HCR chains and barcoding probes (FIG. 6-8). Another distinction of cycleHCR is the introduction of a barcoding phase utilizing pairs of 14bp Left (L) and Right (R) DNA barcoding probes equipped with split HCR initiators (FIG. 1A). These probes are designed to trigger HCR reactions only when perfectly matched to the barcode on the target (FIG. 1B), achieving high-specificity target recognition both in cultured cells and thick tissue specimens (FIG. 1B, 1C and 1E). Through the combination of 30 unique L and 30 unique R probes per color channel, cycleHCR facilitates the generation of up to 900 distinct barcodes, thereby enabling the potential encoding of 2,700 targets across three channels (FIG. 1D).

The use of highly stable primary probes and precise barcoding negates the need for primary probe removal and rehybridization during multicycle HCR imaging. Through optimization, a consistent temperature of 32° C. has been determined to allow for signal removal in 20 minutes (FIG. 8) and HCR amplification in 1.5 hours, achieving about 51% of the signal intensity of overnight amplification. Efficient barcoding is accomplished in under 30 minutes using high concentrations (150˜200 nM) of L+R probes at this temperature. These optimizations collectively reduce the duration of one detection cycle to within 4 hours. Additionally, employing an oxygen scavenger imaging buffer prevents photo-crosslinking during imaging (FIG. 9), maintaining detection fidelity across multiple imaging cycles. Systematic validation showed negligible cross-talks between L+R barcodes at 32° C. (FIG. 10 and FIG. 11A). Nuclear volumes remained consistent over 40 imaging cycles, indicating minimal sample deformation (FIG. 11B). Further analysis revealed that HCR amplification at this temperature with 1.5 hours achieved 103.7-, 88.3-, and 121.3-fold amplification for the 488, 561, and 640 channels, respectively (FIG. 12). Gene expression measurements using two distinct codebooks showed high correlation (r=0.93) (FIG. 1F), validating the linearity and reliability of cycleHCR detection.

Transitioning towards full automation, our setup incorporates a programmable robotic arm for precise preparation of L+R readout mixes (FIG. 7C), and an automated imaging and fluidic system capable of executing cycleHCR protocols and image acquisition without the need of human supervision (FIG. 7B and 7D). This setup supports up to five imaging rounds analyzing 15 RNA species daily. Consistency and reproducibility in data analysis are facilitated by a scalable and portable Nextflow image processing workflow that manages essential tasks such as image stitching, cross-cycle registration, single-molecule localization, 3D cell segmentation, and spot-to-cell assignment (FIG. 13).

For high-resolution imaging of thick specimens, our setup utilizes spinning disk confocal microscopy equipped with silicone oil immersion objectives that offer long working distances up to 550 μm. Integrating hydrogel-embedding and enzymatic digestion-based tissue clearing with cycleHCR allows for detailed 3D visualization of complex sub-cellular RNA patterns within thick tissues. For instance, within a ˜200μm section of mouse cerebellum, we observed dense Slc1a3 mRNA clusters in Bergmann glia, Rgs8 gene transcriptional bursting sites in Purkinje nuclei (FIG. 13C), and Rgs8 transcripts along dendrites. Interestingly, about half of Purkinje cells showed two Rgs8 bursting sites, with no cell exceeding this number. This observation hints at a diploid genome within Purkinje cells, offering insights into the discussion regarding their ploidy and genomic constitution.

### Example 21

### Whole-Embryo Transcriptomics Imaging

To explore gene regulation and cell-fate determination in early development, we focused on an intact E6.5-7.0 mouse embryo, requiring ˜310 μm axial coverage. Leveraging cycleHCR, we performed whole-mount transcriptomics imaging of 254 lineage-specific genes through 85 imaging cycles over 20 days, achieving edge-to-edge imaging clarity and single-molecule detection sensitivity (FIG. 2A-D). The synergy between high system stability and robust image registration enables precise 3D alignment of images throughout imaging cycles, ensuring the reliable spatial decoding of RNA distributions. The resulting single-shot images revealed distinct mRNA localization patterns, highlighting the complex genetic interplay underlying early embryogenesis.

Our automated system standardizes cycleHCR reaction and imaging conditions, ensuring consistent RNA molecule detection across cycles and color channels using RS-FISH (FIG. 2D). For single-cell gene expression analysis, we utilized Cellpose for 3D nucleus segmentation, applying a specialist model trained on both xy and yz planes and refined through size filtering, reaching ˜87.6% initial accuracy. Manual curation further refined segmentation, identifying ˜11,029 nuclei. After evaluating various spot-to-cell assignment methods, we adopted the most conservative approach, assigning spots based on Cellpose masks which are slightly dilated compared to nuclei (FIG. 2E). This strategy only captures spots in proximity to or within the nucleus and minimizes contamination for accurate 3D gene expression mapping at the single-cell level, paving the way for in-depth analysis.

### Example 22

### 3D Cell-Fate Mapping

After initial filtering, we identified 186 actively transcribed genes and excluded 484 cells with low gene expression levels (FIG. 14). Uniform Manifold Approximation and Projection (UMAP) analysis on these genes revealed 9 distinct cell clusters (C1-9) (FIG. 3A), expanding from ˜6 clusters previously recognized through single-cell sequencing at this developmental stage. These clusters span a wide range of cell counts, from 286 in the smallest cluster (C6) to 3,223 in the largest (C1), leaving only 876 cells unallocated. Gene clustering, UMAP imputation, and 3D spatial gene expression visualization showed extensive gene expression overlaps among clusters (FIG. 3A), indicating a complex genetic basis for cell-fate specification. For instance, the well-known primitive streak marker gene T was found to be over-represented in clusters C2, C3, and C4, with the highest enrichment observed in C4.

Preliminary lineage assignment, based on marker gene analysis with comparison to previous studies suggests C1 as the epiblast (Pim2, Dnmt3b and Prom1), C2 as the primitive streak (Eomes, Axin2 and Mcm5), C3 as nascent mesoderm (Lefty2, Mixl1 and Frzb), C4 as a cluster of mix lineages (e.g. T (primitive streak and allantois progenitors (24)), Tbx3 (allantois progenitors) and F2r (blood progenitors)), C5 as extraembryonic ectoderm (Tex19.1, Ahnak and Tead4), C6 as anterior visceral endoderm (Cer1 and Dkk1), C7, C9 as diverse endoderm lineages (Col4a1, Sox17, Cubn, Gata4 and Cldn6) and C8 as putative endothelial cells (Cdh5 and Vim).

To gain insights into the spatial organization and developmental context of these transcriptionally defined clusters, we proceeded to map them back to their 3D spatial locations within the embryo, revealing well-organized structures (FIG. 3B). Specifically, the 3D map shows the anatomical segregation of interior clusters—C1 (epiblast), C2 (primitive streak), C3 (nascent mesoderm), C4, and C5 (extraembryonic ectoderm)-into embryonic (C1, C2, C3) and extraembryonic (C5) domains, with C4 serving as a demarcation between the two domains. Within the embryo, the epiblast (C1), primitive streak (C2), and nascent mesoderm (C3), arrange into distinct, symmetrical layers across the midline. The endoderm clusters, C6, C7 and C9, also demonstrate spatial segregations; with C6 encasing the embryonic clusters (C1, C2, C3), C7 encircling the extraembryonic ectoderm (C5) and cells from C9 sparsely surround C6 and C7. Adding to this complex structure, the C8 covers endoderm layers, forming concentric rings.

The anatomical arrangement of identified clusters further refines the interpretation of potential cell fates. For example, the spatial positioning of C1 (Epiblast), C2 (Primitive Streak) and C3 (Nascent mesoderm) aligns with established developmental processes at this stage, including gastrulation and mesoderm migration. Notably, C4, positioned between embryonic and extraembryonic domains, likely represents allantois and blood progenitors destined to form the umbilical cord connecting the fetus to the placenta. The extraembryonic ectoderm (C5) is set to become the placental cone. Gene expression and spatial positioning identify C6 and C7 as visceral endoderm subtypes directly interacting with the embryo, with C6 more concentrated near the epiblast and C7 laterally positioned and enriched in the extraembryonic region. C9, lacking direct embryo contact and with highly specific expression of the gene encoding extracellular matrix protein-Col4a1, likely represents the parietal endoderm, eventually contributing to the parietal yolk sac. The putative endothelial cells (C8) likely mark emerging blood vasculature, crucial for supporting both embryonic and extraembryonic structures. The precise enumeration and positioning of minor or nascent cell groups, such as C4, C6 and C9, highlight the exceptional sensitivity of cycleHCR for in situ spatial analysis of cell types in intact specimens.

### Example 23

### cycleHCR Protein Imaging

To harness the high specificity of cycleHCR for multiplex protein imaging, we have developed an antibody complex that simultaneously recognizes the target and anchors a cycleHCR barcode within a polyacrylamide gel for imaging readout (FIG. 4A). Specifically, the in vitro assembly begins by attaching a docking sequence to the antibody using a light-activated oYo linker, followed by annealing a gel anchoring probe that binds through DNA complementarity. The gel anchoring probe carries a cycleHCR barcode and a 5′ acrydite group for gel integration. Once anchored, the barcode permanently incorporates into the gel matrix, eliminating the need for the presence of the antibody. We have developed a unique set of 25 Left and 25 Right protein cycleHCR barcodes, orthogonal to our RNA-focused set, enabling potential joint imaging of RNA and proteins. This method facilitates precise, antibody-based protein detection over multiple cycleHCR rounds in cultured cells and mouse hippocampal slices (FIG. 4A).

Embedding barcoding oligos into the gel preserves spatial information during later processing steps, making cycleHCR ideal for coupling with tissue clearing and expansion microscopy. To integrate protein cycleHCR imaging with expansion microscopy, we stabilize the expansion gel with a second, non-expandable gel overlay. This ensures stable imaging and reliable image alignment through multiple cycles, allowing us to capture high-resolution images of 10 subcellular structures in primary mouse embryonic fibroblasts (FIG. 4B). 3D segmentation reveals the complex spatial organization of these structures (FIG. 4B), including distinct nuclear regions enriched for heterochromatin, nucleolar fibrillar centers, nuclear speckles, and active enhancers. These results highlight the potential utility of cycleHCR protein imaging in characterizing subcellular structures and their interrelations. See, also, FIGS. 16-17.

### Example 24

### Multiplex RNA and Protein Imaging Via cycleHCR

Next, we investigated the potential of cycleHCR technology for multiplex RNA and protein imaging to elucidate cell-fate map and sub-cellular structures within the hippocampal slice. A library targeting 120 genes that label diverse cell types in the hippocampus was constructed. A 40-60 μm thick hippocampal slice was first stained with 8 cycleHCR barcoded antibodies. After gel embedding, tissue clearing, and hybridization of the cycleHCR RNA library, 44 readout cycles were conducted over 11 days, with 40 cycles targeting RNA and 4 cycles targeting proteins. High system stability and precise cross-round image registration ensured high-quality visualization of RNA and protein signals through cycleHCR rounds (FIG. 5A-D). Accurate 3D cell segmentation and spot identification facilitated single-cell gene expression analysis. Utilizing UMAP-based clustering, we uncovered 6 transcriptionally and spatially segregated clusters (denoted as C1-C6), corresponding to distinct neuronal and glial cell types (FIG. 5E; FIG. 15). Notably, we observed complex gene expression gradients along the middle line of C1-C2-C3, with 33genes displaying descending expression levels and 7 genes showing ascending levels (FIG. 5F-G). The unified cycleHCR readout of RNA and protein compositions enabled cell-type characterization and sub-cellular structural investigation within the same specimen. This approach unveiled cluster-specific nuclear structural variances (FIG. 5H), suggestive of a potential link between cell fate and nuclear architectures.

### Example 25

### Composition of RNA-Targeting Primary Probes with Split Combinatory DNA Barcode

To target each RNA sequence with high specificity, we engineered DNA probe libraries with 10 to 25 pairs of 45-bp probes. Each pair consists of left and right probes with unique 14-bp barcode sequences for binding left (L) and right (R) readout probes with split HCR initiators, triggering the hybridization chain reaction (HCR). The arrowhead indicates the 3′ end of the RNA or DNA. See FIG. 1A.

### Ingredients:

Extended left and right primary probes (45 bp each) with a high melting temperature on RNA (>90° C.), ensuring stable binding to target RNA in the sample. Split left and right barcode sequences (14 bp each), designed to hybridize specifically to barcode readout probes carrying split HCR initiators.

Each barcode sequence is attached to the extended split primary probe (45 bp each), ensuring stable binding to the target molecule.

The split barcode pairs are complementary to readout probes that carry split HCR initiators, enabling downstream signal amplification for specific target detection.

### Utility:

The high melting temperature allows the primary probes to remain stably bound to RNA during the stripping process, while other components—such as HCR chains and barcoding probes—are efficiently removed.

This enables multiple rounds of barcoding, HCR amplification, imaging, and signal removal, facilitating sequential molecular imaging workflows.

Molecular identity is determined through a unique combinatorial pairing of split left and right barcode sequences, forming a distinct hybridization code for each target.

Readout probes carry split HCR initiators hybridize selectively to specific barcode pairs, triggering HCR amplification only when both barcode halves are in proximal binding, thus: 1) Reducing off-target interactions by requiring co-localization of barcode components. 2) Enhancing labeling specificity, ensuring that signal amplification occurs only at the intended target.

The split combinatorial design enables highly multiplexed molecular profiling. Key Points:

A split primary probe system with a melting temperature on RNA exceeding that of the HCR chain, ensuring selective retention of the primary probes while allowing removal of other signal amplification components.

Split left and right DNA barcode system that employs a combinatorial approach to determine molecular identity.

Barcode designs that require both split left and right barcode halves to be present for downstream proximity-dependent signal amplification, improving labeling specificity.

Compatibility with hybridization chain reaction (HCR) amplification, fluorescence imaging, and other molecular detection methods.

### Example 26

### Composition of Barcode Readout Probes Carrying Split HCR Initiators

To target each RNA sequence with high specificity, we engineered DNA probe libraries with 10 to 25 pairs of 45-bp probes. Each pair consists of left and right probes with unique 14-bp barcode sequences for binding left (L) and right (R) readout probes with split HCR initiators, triggering the hybridization chain reaction (HCR). The arrowhead indicates the 3′ end of the RNA or DNA. See FIG. 1A.

Using 30 unique L and 30 unique R probes per color channel, cycleHCR facilitates the generation of up to 900 distinct barcodes, thereby enabling the potential encoding of 2,700 targets across three channels.

### Ingredients:

14 bp barcode readout probes, each designed to hybridize specifically to a split DNA barcode on RNA or protein targets.

Each readout probe carries a split HCR initiator, which remains inactive until the correct hybridization event occurs.

### Utility:

A specific left and right pair of readout probes binds to a split DNA barcode on the target molecule (RNA or protein).

Upon hybridization, the split HCR initiator is reconstituted, enabling high-specificity activation of downstream HCR amplification.

The proximity-dependent activation minimizes non-specific background amplification and enhanced signal specificity, improving molecular detection accuracy.

The design supports highly multiplexed molecular profiling, allowing for precise spatial mapping of target molecules.

### Key Points:

A barcode readout probes carrying a split HCR initiator.

Barcode readout probe designs that require both left and right components to hybridize to a split barcode before reconstituting the HCR initiator.

Compatibility with hybridization chain reaction (HCR) amplification, fluorescence imaging, and other molecular detection methods.

### Example 27

### Composition of Gel Anchoring Probes

cycleHCR protein labeling involves in vitro tri-functional antibody complex assembly. This method covalently attaches a docking sequence (green) to the antibody using the oYo linker. The sequence is then hybridized with a gel anchoring probe that contains a L+R cycleHCR barcode (light orange and blue) and a 5′ acrydite group for attachment to the polyacrylamide gel. See FIG. 4A.

Using 60 unique L and 60 unique R probes per color channel, cycleHCR facilitates the generation of up to 3,600 distinct barcodes, thereby enabling the potential encoding of 10,800 antibodies across three channels. These barcodes are distinct from and orthogonal to the 2,700 RNA barcodes, enabling joint RNA and protein imaging in the same sample.

### Ingredients:

The gel anchoring probes are composed of three key components:

### Component 1—Gel Anchoring Group:

5′ acrydite group for covalent incorporation into the polyacrylamide gel, ensuring stable anchoring.

### Component 2—Antibody Docking Sequence:

An 18-20 bp sequence designed to bind to a docking sequence on the antibody, enabling targeted protein labeling.

Component 3—Split Combinatory DNA Barcode:

A split DNA barcode that can be recognized by specific pairs of barcode readout probes, which carry initiators for triggering downstream HCR amplification.

### Utility:

This specific composition enables in vitro antibody complex assembly by utilizing:

A docking sequence on the antibody for precise probe attachment.

Acrydite-based gel anchoring, ensuring that the split combinatory DNA barcode remains immobilized within the polyacrylamide matrix, enabling multi-round detection by resisting stripping.

The anchored split barcode system allows HCR amplification to be triggered with high specificity.

This design facilitates multiplexed protein detection, making it ideal for gel-based molecular profiling workflows.

### Key Points:

A probe containing a combination of:
5′ acrydite group for gel incorporation.
split combinatory DNA barcode for molecular recognition and signal amplification.

compatibility with HCR-based signal amplification, fluorescence imaging, and spatially resolved proteomic detection methods.

### Example 28

### Discussion

Biological systems are complex networks of interconnected components, making it crucial to understand their interrelationships within intact tissue environments. Conventional fluorescence microscopy, constrained by fluorescent dye limitations, only allows for the observation of a limited number of components, limiting our comprehension of complex spatial regulations. Deep tissue imaging further faces challenges such as high autofluorescence and the necessity for objectives with low numerical apertures and long working distances, which are not well-suited for detecting dim single molecules. Existing imaging based spatial omics techniques require barcoding at the single-molecule level, which, combined with the need for cross-round registration, poses significant challenges in identifying targets with variable densities in thick specimens. As a result, these techniques are largely limited to thin tissue sections, leading to a loss of three-dimensional context.

The development of split HCR systems enhances deep tissue imaging by providing high-specificity target recognition and significant signal amplification. Built on these achievements, we introduce cycleHCR technology, which utilizes DNA barcoding to enable unified high-throughput imaging of both RNA and proteins. This approach leverages high system stability and robust image registration, ensuring accurate image alignment through multiple cycles. This synergy allows cycleHCR to map spatial information of molecular compositions and subcellular structures over successive imaging cycles, effectively overcoming the color barrier. The capability of cycleHCR to pinpoint and resolve minor or nascent cell groups underscores its significance in developmental biology and oncology research. Furthermore, the integration of RNA and protein imaging in a single platform, along with its compatibility with expansion microscopy, positions cycleHCR as a tool for both detailed transcriptomic studies and the characterization of high-resolution molecular architectures. This integrated approach bridges the gap between identifying cell types and understanding their sub-cellular arrangements, offering an integral view of complex biological systems.

The comparison of cycleHCR to other spatial omics methods brings to light both its unique advantages and its primary limitation, which is the lower throughput. This limitation arises mainly because of the Hybridization Chain Reaction (HCR) amplification process and its reliance on a single-shot approach for each target. However, cycleHCR uniquely bypasses the issue of molecular crowding, as it generates empirical images for both RNA and protein species without requiring spot-level registration and complex decoding between rounds. This is a significant advantage in studies where preserving the spatial integrity and avoiding the complications of molecular crowding are crucial.

Furthermore, the HCR amplification inherent to cycleHCR enhances signal brightness and contrast against background. This feature allows for the use of low-aperture objectives for deep imaging and rapid acquisition over large volumes. This capability is particularly valuable for studying thick tissue samples or whole organisms where depth penetration and volume imaging are required. Future improvements in imaging depth are expected by using longer-working-distance water immersion objectives. Additionally, light-sheet microscopy, known for its efficient illumination and reduced photobleaching, could complement cycleHCR, enabling deeper imaging while preserving high resolution and reducing sample damage, further enhancing its utility.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. An article of manufacture comprising:
a plurality of pairs of primary probes, wherein each pair of primary probes comprises left primary probe and a right primary probe, wherein: the left primary probe comprises a forward PCR sequence, a left target complementary sequence, a left barcode sequence, and a reverse PCR sequence, and the right primary probe comprises the forward PCR sequence, a right barcode sequence, a right target complementary sequence, and the reverse PCR sequence;
a plurality of pairs of readout probes, wherein each pair of readout probes comprises left readout probe and a right readout probe, wherein the left readout probe comprises a left HCR initiation sequence and a sequence that is complementary to the left barcode sequence and the right readout probe comprises a right HCR initiation sequence and a sequence that is complementary to the right barcode sequence.

2. The article of manufacture of claim 1, further comprising a first and a second HCR amplifier sequence, wherein each of the first and the second HCR amplifier sequences comprise a sequence that forms a hairpin and a detectable label, wherein a portion of the first HCR amplifier sequence and a portion of the second HCR amplifier sequence have complementarity to one another.

3. The article of manufacture of claim 1, further comprising reagents necessary for hybridization chain reaction (HCR) to occur.

4. The article of manufacture of claim 1, further comprising one or more detectable labels (e.g., fluorophores or pairs of fluorophores).

5. The article of manufacture of claim 1, further comprising one or more reagents necessary for fixing the biological specimen.

6. The article of manufacture of claim 1, further comprising one or more reagents necessary for stripping HCR products and probes from the biological specimen.

7. A method of spatially mapping a target RNA in a biological specimen, comprising:
contacting the biological specimen with a pair of primary probes, wherein the pair of primary probes comprises a left primary probe and a right primary probe, wherein:
the left primary probe comprises a forward PCR sequence, a left target complementary sequence, a left barcode sequence, and a reverse PCR sequence, and
the right primary probe comprises the forward PCR sequence, a right barcode sequence, a right target complementary sequence, and the reverse PCR sequence;
contacting the biological specimen with a pair of readout probes, wherein the pair of readout probes comprises a left readout probe and a right readout probe, wherein:
the left readout probe comprises a left HCR initiation sequence and a sequence that is complementary to the left barcode sequence and
the right readout probe comprises a right HCR initiation sequence and a sequence that is complementary to the right barcode sequence;
contacting the biological specimen with a first and a second HCR amplifier sequence, wherein each of the first and the second HCR amplifier sequences comprise a sequence that forms a hairpin and a detectable label, wherein a portion of the first HCR amplifier sequence and a portion of the second HCR amplifier sequence have complementarity to one another;
exposing the biological specimen to conditions under which hybridization chain reaction (HCR) occurs to produce labeled HCR products; and
imaging the labeled HCR products in the biological specimen, thereby spatially mapping the target RNA in the biological specimen.

8. The method of claim 7, wherein the biological specimen is cultured cells or tissue.

9. The method of claim 7, wherein the biological specimen is fixed.

10. The method of claim 7, wherein the biological specimen is permeabilized, gelled, contacted with a protease, stained (e.g., DAPI), washed, or combinations thereof.

11. The method of claim 7, further comprising stripping the HCR products and the readout probes from the biological specimen and repeating the contacting and exposing steps with a different pair of readout probes.

12. The method of claim 7, wherein the biological specimen is contacted with a plurality of pairs of primary probes.

13. The method of claim 7, wherein the plurality of pairs of primary probes is comprised within a primary probe library.

14. The method of claim 7, wherein the detectable label is a fluorophore or one member of a pair of fluorophores.

15. The method of claim 7, further comprising evaluating the quality of the RNA in the biological specimen.

16. The method of claim 7, wherein the spatially mapping is three-dimensional.

17. The method of claim 7, wherein the method is high-throughput.

18. The method of claim 7, wherein the method is fully automated.

19. An article of manufacture comprising
a pair of docking sequences comprising a first docking sequence and second docking sequence, wherein at least a portion of the first docking sequence and the sequence docking sequence is identical or essentially identical; and
a pair of gel anchoring probes comprising a first gel anchoring probe and a second gel anchoring probe, wherein a portion of the first gel anchoring probe and second gel anchoring probe are complementary to the portion of the first and second docking sequence that are identical or essentially identical, wherein the first gel anchoring probe and the second gel anchoring probe each comprise a right barcode sequence and a left barcode sequence.

20. The article of manufacture of claim 19, further comprising a pair of readout probes, wherein the pair of readout probes comprises a left readout probe and a right readout probe, wherein the left readout probe comprises a left HCR initiation sequence and a sequence that is complementary to the left barcode sequence and the right readout probe comprises a right HCR initiation sequence and a sequence that is complementary to the right barcode sequence.

21. The article of manufacture of claim 19, further comprising a first and a second HCR amplifier sequence, wherein each of the first and the second HCR amplifier sequences comprise a sequence that forms a hairpin and a detectable label, wherein a portion of the first HCR amplifier sequence and a portion of the second HCR amplifier sequence have complementarity to one another.

22. The article of manufacture of claim 19, further comprising one or more proteins that bind one or more targets in the biological specimen.

23. The article of manufacture of claim 19, further comprising reagents necessary for hybridization chain reaction (HCR) to occur.

24. The article of manufacture of claim 19, further comprising one or more detectable labels (e.g., fluorophores or pairs of fluorophores).

25. The article of manufacture of claim 19, further comprising one or more reagents necessary for fixing the biological specimen.

26. The article of manufacture of claim 19, further comprising one or more reagents necessary for stripping HCR products and probes from the biological specimen.

27. The article of manufacture of claim 19, further comprising a linker.

28. The article of manufacture of claim 27, wherein the linker is a light-activated oYo linker.

29. The article of manufacture of claim 19, wherein the pair of gel anchoring probes further comprises at least one 5′ acrydite modifications.

30. A method of spatially mapping a target protein in a biological specimen, comprising:
contacting the biological specimen with an antibody complex that binds specifically to the target protein, wherein the antibody complex comprises two identical or essentially identical docking sequences covalently attached thereto, wherein the antibody complex further comprises two identical or essentially identical gel anchoring probes hybridized to the two docking sequences, wherein each of the gel anchoring probes further comprises a left barcode sequence and a right barcode sequence;
immobilizing the biological specimen comprising the antibody complex bound to the target protein in a gel via either or both of the gel anchoring probes;
contacting the biological specimen with a pair of readout probes, wherein the pair of readout probes comprises a left readout probe and a right readout probe, wherein the left readout probe comprises a left HCR initiation sequence and a sequence that is complementary to the left barcode sequence and the right readout probe comprises a right HCR initiation sequence and a sequence that is complementary to the right barcode sequence;
contacting the biological specimen with a first and a second HCR amplifier sequence, wherein each of the first and the second HCR amplifier sequences comprise a sequence that forms a hairpin and a detectable label, wherein a portion of the first HCR amplifier sequence and a portion of the second HCR amplifier sequence have complementarity to one another;
exposing the biological specimen to conditions under which hybridization chain reaction (HCR) occurs to produce HCR products; and
imaging the HCR products in the biological specimen, thereby spatially mapping the target protein in the biological specimen.

31. The method of claim 30, wherein the biological specimen is cultured cells or tissue.

32. The method of claim 30, wherein the biological specimen is fixed.

33. The method of claim 30, wherein the biological specimen is permeabilized, gelled, contacted with a protease, staining, washing, and combinations thereof.

34. The method of claim 30, wherein the docking sequence is attached to the antibody via a linker.

35. The method of claim 34, wherein the linker is a light-activated oYo linker.

36. The method of claim 30, wherein the gel anchoring probes comprise at least one 5′ acrydite modifications.

37. The method of claim 30, further comprising stripping the HCR products and the readout probes from the biological specimen and repeating the contacting and exposing steps with a different pair of readout probes.

38. The method of claim 30, wherein the method is high-throughput.

39. The method of claim 30, wherein the method is fully automated.
